(19) United States
(12) Patent Application Publication
(10) Pub. No.: US 2025/0028164 A1
(43) Pub. Date: Jan. 23, 2025

(54) LIGHT SOURCE DEVICE AND SYSTEM

(71) Applicant: FUJIFILM Corporation, Tokyo (JP)

(72) Inventors: Takeshi FUKUDA, Ashigarakami-gun (JP); Makoto SUGIZAKI, Ashigarakami-gun (JP)

(73) Assignee: FUJIFILM Corporation, Tokyo (JP)

(21) Appl. No.: 18/778,339

(22) Filed: Jul. 19, 2024

(30) Foreign Application Priority Data
Jul. 21, 2023 (JP) 2023-119244

(51) Int. Cl. G02B 23/24; A61B 1/00; A61B 1/06

(52) U.S. Cl. CPC G02B 23/2461; A61B 1/00177; A61B 1/0638; A61B 1/0684

(57) ABSTRACT

A system includes: a light source device to which a plurality of types of endoscopes as defined herein are connectable; and a processor, the light source device includes: a first light source; a second light source; a third light source; a fourth light source; and an optical member, the fourth light source generates the white light as defined herein, an optical path of light emitted from the fourth light source to the light guide is shorter than optical paths of light emitted from the first light source, the second light source and the third light source to the light guide, and the processor is configured to perform white balance processing on a captured image signal output from an imaging element of one of the endoscopes connected to the light source device based on a type of the one of the endoscopes.

### CROSS-REFERENCE TO RELATED APPLICATIONS

The present application claims priority under 35 U.S.C. § 119 to Japanese Patent Application No. 2023-119244, filed on Jul. 21, 2023. The above application is hereby expressly incorporated by reference, in its entirety, into the present application.

### BACKGROUND OF THE INVENTION

### 1. Field of the Invention

The present invention relates to a light source device and a system.

### 2. Description of the Related Art

CN109507794A, CN209570749Y, CN111110175A, and CN212521711Y describe a light source device of an endoscope.

JP4795202B, JP4160577B, and JP2018-99441A describe various types of endoscopes.

JP2019-508A, WO2021/065939A, and JP1994-17942B (JP-H06-17942B) describe a technology related to white balance processing of an endoscope.

### SUMMARY OF THE INVENTION

The present disclosure provides a technology capable of improving quality of a captured image of an endoscope.

According to one aspect of the technology of the present disclosure, there is provided a system comprising: a light source device to which a plurality of types of endoscopes including light guides with different outer diameters are connectable; and a processor, in which the light source device includes: a first light source that includes a light emitting diode and that generates red light; a second light source that includes a light emitting diode and that generates blue light; a third light source that includes a light emitting diode and that generates violet light; a fourth light source that includes a light emitting diode and that generates white light; and an optical member that is capable of introducing at least two of the red light, the blue light, the violet light, and green light included in the white light into the light guide of the endoscope by combining the at least two, the fourth light source generates the white light by using a blue light emitting diode that generates blue light and a phosphor that generates light by receiving the blue light, an optical path of light emitted from the fourth light source to the light guide is shorter than an optical path of light emitted from the first light source, the second light source, and the third light source to the light guide, and the processor performs white balance processing on a captured image signal output from an imaging element of an endoscope connected to the light source device based on a type of the endoscope.

According to one aspect of the technology of the present disclosure, there is provided a light source device comprising: a first light source that includes a light emitting diode and that generates red light; a second light source that includes a light emitting diode and that generates blue light; a third light source that includes a light emitting diode and that generates violet light; a fourth light source that includes a light emitting diode and that generates white light; an optical member that is configured to introduce the red light into a light guide of an endoscope, introduce the blue light into the light guide, introduce the violet light into the light guide, and introduce green light included in the white light into the light guide; a detection unit that detects a part of light generated by at least two of the first light source, the second light source, the third light source, and the fourth light source; and a processor that controls amounts of light generated by at least two of the first light source, the second light source, the third light source, and the fourth light source based on the light detected by the detection unit.

According to one aspect of the technology of the present disclosure, there is provided a light source device comprising: a first light source that includes a light emitting diode and that generates first color light; a second light source that includes a light emitting diode and that generates second color light; a third light source that includes a light emitting diode and that generates violet light; a fourth light source that includes a light emitting diode and that generates light including at least green light; a first combining member that is capable of combining the first color light and the second color light; a second combining member that is capable of combining light emitted from the first combining member and one of the violet light and the green light; a third combining member that is capable of combining light emitted from the second combining member and the other of the violet light and the green light; a condensing member that condenses light emitted from the third combining member into a light guide of an endoscope; a first detection unit that detects a part of light emitted from the second combining member and incident on the third combining member and a part of light emitted from the fourth light source and incident on the third combining member; and a processor that controls amounts of light generated by at least two of the first light source, the second light source, the third light source, and the fourth light source based on the light detected by the first detection unit.

According to the technology of the present disclosure, it is possible to improve quality of a captured image of an endoscope.

### BRIEF DESCRIPTION OF THE DRAWINGS

FIG. 1 is a diagram showing a schematic configuration of an endoscope apparatus 100 which is one aspect of the technology of the present disclosure.

FIG. 2 is a schematic diagram showing an internal configuration of a light source device 5.

FIG. 3 is a schematic diagram illustrating a configuration of a fourth light source 54.

FIG. 4 is a schematic diagram illustrating a modification example of the fourth light source 54.

FIG. 5 is a diagram showing a light source device 5B which is a first modification example of the light source device 5.

FIG. 6 is a diagram showing a light source device 5C which is a second modification example of the light source device 5.

FIG. 7 is a schematic diagram illustrating a configuration of a fourth light source 54Xa which is a modification example of a fourth light source 54X in FIG. 6.

FIG. 8 is a diagram showing a light source device 5D which is a third modification example of the light source device 5.

FIG. 9 is a diagram showing a light source device 5F which is a fourth modification example of the light source device 5.

FIG. 10 is a diagram showing a light source device 5H which is a fifth modification example of the light source device 5.

FIG. 11 is a schematic diagram showing a configuration example of a detection unit 60 in the light source device 5.

FIG. 12 is a schematic diagram showing a configuration example of the detection unit 60 in the light source device 5C.

FIG. 13 is a schematic diagram showing a configuration example of the detection unit 60 in the light source device 5C.

FIG. 14 is a schematic diagram showing a configuration example of the detection unit 60 in the light source device 5B.

FIG. 15 is a diagram showing a modification example of the light source device 5 shown in FIG. 11.

FIG. 16 is a schematic diagram showing a configuration example of the detection unit 60 in the light source device 5D.

FIG. 17 is a schematic diagram showing a configuration example of the detection unit 60 in the light source device 5F.

FIG. 18 is a schematic diagram showing a configuration example of the detection unit 60 in the light source device 5F.

FIG. 19 is a schematic diagram showing a configuration example of the detection unit 60 in the light source device 5H.

FIG. 20 is a schematic diagram showing a configuration example of the detection unit 60 in the light source device 5.

FIG. 21 is a schematic diagram showing a configuration example of the detection unit 60 in the light source device 5.

FIG. 22 is a schematic diagram showing a configuration example of a detection unit 60 in the light source device 5.

FIG. 23 is a schematic diagram showing a configuration example of the detection unit 60 in the light source device 5.

FIG. 24 is a schematic diagram showing a configuration example of the detection unit 60 in the light source device 5.

FIG. 25 is a schematic diagram showing a configuration example of the detection unit 60 in the light source device 5.

### DESCRIPTION OF THE PREFERRED EMBODIMENTS

In the present specification, violet light refers to light in a wavelength range of 380 nm or more and 430 nm or less. Blue light refers to light in a wavelength range of 430 nm or more and 490 nm or less. Green light refers to light in a wavelength range of 490 nm or more and 560 nm or less. Yellow light refers to light in a wavelength range of 560 nm or more and 600 nm or less. Red light refers to light in a wavelength range of 600 nm or more and 650 nm or less.

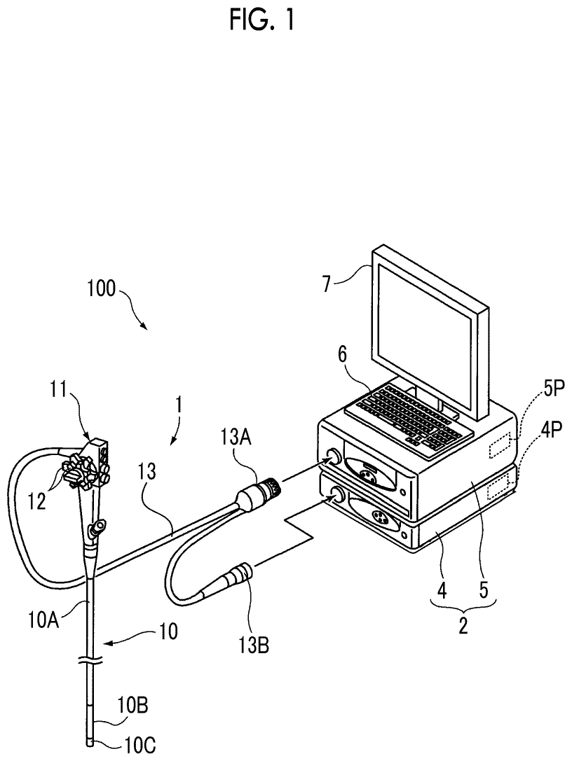

FIG. 1 is a diagram showing a schematic configuration of an endoscope apparatus 100 which is one aspect of the technology of the present disclosure.

The endoscope apparatus 100 constitutes a system. The endoscope apparatus 100 comprises an endoscope 1, a body part 2 that consists of a processor device 4 and a light source device 5 to which the endoscope 1 is connected, a display device 7 that displays a captured image obtained by imaging with the endoscope 1, and the like, and an input unit 6 that is an interface for inputting various types of information to the processor device 4.

The endoscope 1 comprises an insertion part 10 that is an elongated instrument extending in one direction and that is inserted into a subject, an operation part 11 that is provided at a proximal end part of the insertion part 10 and that is provided with an operation member for performing operations such as an observation mode switching operation, an imaging storing operation, a forceps operation, an air and water supply operation, a suction operation, or an electric cautery operation, an angle knob 12 provided adjacent to the operation part 11, and a universal cord 13 that includes connector portions 13A and 13B which attachably and detachably connect the endoscope 1 to the light source device 5 and to the processor device 4, respectively.

The operation part 11 is provided with a forceps port into which a biopsy forceps, which is a treatment tool for collecting a biological tissue, such as a cell or a polyp, is inserted. Although not shown in FIG. 1, various channels such as a forceps channel through which the biopsy forceps inserted from the forceps port is inserted, a channel for air and water supply, and a channel for suction are provided inside the operation part 11 and the insertion part 10.

The insertion part 10 is composed of a flexible soft portion 10A, a bending portion 10B provided at a distal end of the soft portion 10A, and a distal end portion 10C that is harder than the soft portion 10A and that is provided at a distal end of the bending portion 10B. An imaging element and an imaging optical system are incorporated into the distal end portion 10C. The imaging element includes a light-receiving surface on which, for example, a first pixel for detecting blue light and violet light, a second pixel for detecting red light, and a third pixel for detecting green light are two-dimensionally arranged.

The bending portion 10B is configured to bend by a rotational movement operation of the angle knob 12. The bending portion 10B can bend in any direction and at any angle depending on a site of the subject being examined with the endoscope 1, allowing the distal end portion 10C to be directed in a desired direction.

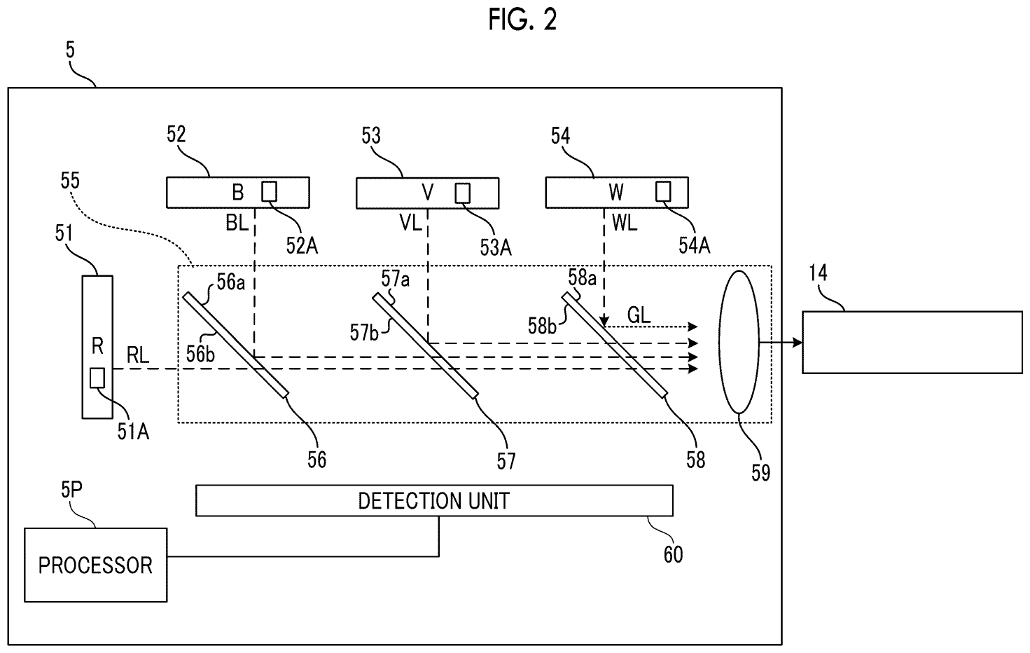

Inside the endoscope 1, a light guide 14 (see FIG. 2) composed of a plurality of optical fibers bundled from the distal end portion 10C of the insertion part 10 to the connector portion 13A is provided. Light generated by the light source device 5 is introduced from the connector portion 13A into the light guide 14, advances to the distal end portion 10C, and is emitted to the subject from an illumination window provided at the distal end portion 10C.

The processor device 4 comprises a processor 4P that controls the endoscope 1, the light source device 5, and the display device 7, and a memory composed of storage media such as a random-access memory (RAM), a read-only memory (ROM), a solid-state drive (SSD), or a hard disk drive (HDD). The light source device 5 comprises a processor 5P that controls a light source, which will be described below, and a memory.

The processor 4P and the processor 5P are each a central processing unit (CPU) that is a general-purpose processor which executes software to perform various functions, a programmable logic device (PLD) that is a processor of which a circuit configuration can be changed after manufacture, such as a field-programmable gate array (FPGA), a dedicated electrical circuit that is a processor having a dedicated circuit configuration designed to execute specific processing, such as an application-specific integrated circuit (ASIC), or the like.

The processor 4P and the processor 5P may be each configured with one processor or a combination of two or more processors of the same or different types (for example, a combination of a plurality of FPGAs or a combination of a CPU and an FPGA). A hardware structure of each of the processor 4P and the processor 5P is more specifically an electrical circuit (circuitry) in which circuit elements such as semiconductor elements are combined.

FIG. 2 is a schematic diagram showing an internal configuration of the light source device 5.

The light source device 5 comprises a first light source 51, a second light source 52, a third light source 53, a fourth light source 54, an optical member 55, and a detection unit 60 that detects a part of light generated by at least two of the first light source 51, the second light source 52, the third light source 53, and the fourth light source 54. The first light source 51, the second light source 52, the third light source 53, and the fourth light source 54 are controlled by the processor 5P. The processor 5P controls amounts of light generated by at least two of the first light source 51, the second light source 52, the third light source 53, and the fourth light source 54 based on the light detected by the detection unit 60. Details of the detection unit 60 and the control of the amounts of light will be described below.

The first light source 51 includes a light emitting diode 51A that generates red light. The first light source 51 uses the light emitting diode 51A to generate red light RL having wavelength characteristics, for example, with a central wavelength of 620 nm or more and 640 nm or less and a half-width of 15 nm or more and 30 nm or less.

The second light source 52 includes a light emitting diode 52A that generates blue light. The second light source 52 uses the light emitting diode 52A to generate blue light BL having wavelength characteristics, for example, with a central wavelength of 440 nm or more and 450 nm or less and a half-width of 15 nm or more and 30 nm or less.

The third light source 53 includes a light emitting diode 53A that generates violet light. The third light source 53 uses the light emitting diode 53A to generate violet light VL having wavelength characteristics, for example, with a central wavelength of 410 nm or more and 430 nm or less and a half-width of 15 nm or more and 30 nm or less.

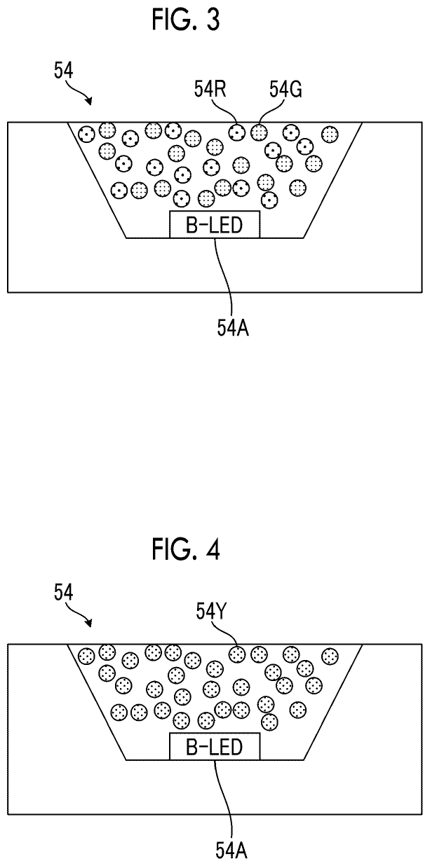

FIG. 3 is a schematic diagram illustrating a configuration of the fourth light source 54.

The fourth light source 54 includes a light emitting diode 54A that generates blue light for excitation, a first phosphor 54R that generates red light by receiving the blue light generated by the light emitting diode 54A, and a second phosphor 54G that generates green light by receiving the blue light generated by the light emitting diode 54A. The fourth light source 54 generates white light WL by mixing the blue light generated by the light emitting diode 54A, the red light generated by the first phosphor 54R, and the green light generated by the second phosphor 54G.

FIG. 4 is a schematic diagram illustrating a modification example of the fourth light source 54.

The fourth light source 54 includes a third phosphor 54Y that generates yellow light by receiving the blue light generated by the light emitting diode 54A, instead of the first phosphor 54R and the second phosphor 54G. As described above, the fourth light source 54 may be configured to generate the white light WL by mixing the blue light generated by the light emitting diode 54A and the yellow light generated by the third phosphor 54Y.

In the configuration shown in FIG. 3, it is possible to enhance color rendering because the red light and the green light can be provided over a broader bandwidth as compared with the configuration shown in FIG. 4. In addition, it is possible to increase an amount of light of the green light on a shorter wavelength side. As shown in FIGS. 3 and 4, by combining the light emitting diode and the phosphor to generate the white light WL, it is possible to inexpensively generate the white light WL including broadband green light.

The optical member 55 is configured to be capable of introducing the red light RL generated by the first light source 51 into the light guide 14 of the endoscope 1, introducing the blue light BL generated by the second light source 52 into the light guide 14, introducing the violet light VL generated by the third light source 53 into the light guide 14, and introducing the green light (hereinafter, referred to as green light GL) included in the white light WL generated by the fourth light source 54 into the light guide 14. The optical member 55 is configured to be capable of introducing at least two of four colors of light, that is, the red light RL, the blue light BL, the violet light VL, and the green light GL, into the light guide 14 by combining the at least two.

In the example of FIG. 2, the optical member 55 comprises three combining members (a combining member 56, a combining member 57, and a combining member 58), each capable of combining a plurality of rays of light, and a condenser lens 59 as a condensing member.

The combining member 56, the combining member 57, and the combining member 58 are each configured to reflect light incident on one surface in a predetermined direction and to transmit light incident on the other surface in the predetermined direction, so as to combine the light incident on the one surface and the light incident on the other surface and to emit the combined light in the predetermined direction. The combining member 56, the combining member 57, and the combining member 58 are each configured with, for example, a dichroic mirror having characteristics of reflecting light having a specific wavelength and of transmitting other light.

In the example of FIG. 2, the first light source 51, the combining member 56, the combining member 57, the combining member 58, and the condenser lens 59 are arranged in this order in a direction along an optical axis of the condenser lens 59 (hereinafter, referred to as an optical axis direction). The combining member 56 is disposed in a state in which one surface 56a and the other surface 56b are angled at 45 degrees with respect to the optical axis of the condenser lens 59. The combining member 57 is disposed in a state in which one surface 57a and the other surface 57b are angled at 45 degrees with respect to the optical axis of the condenser lens 59. The combining member 58 is disposed in a state in which one surface 58a and the other surface 58b are angled at 45 degrees with respect to the optical axis of the condenser lens 59.

With respect to the combining member 56, the second light source 52 is disposed on one side (an upper side in FIG. 2) in a direction perpendicular to the optical axis direction of the condenser lens 59, and the blue light BL generated by the second light source 52 is incident on the one surface 56a of the combining member 56.

With respect to the combining member 57, the third light source 53 is disposed on one side (the upper side in FIG. 2) in a direction perpendicular to the optical axis direction of the condenser lens 59, and the violet light VL generated by the third light source 53 is incident on the one surface 57a of the combining member 57.

With respect to the combining member 58, the fourth light source 54 is disposed on one side (the upper side in FIG. 2) in a direction perpendicular to the optical axis direction of the condenser lens 59, and the white light WL generated by the fourth light source 54 is incident on the one surface 58a of the combining member 58.

The combining member 56 is configured to reflect the blue light BL incident on the one surface 56a in a direction of the combining member 57 and to transmit the red light RL incident on the other surface 56b in the direction of the combining member 57, so as to combine the red light RL and the blue light BL and to emit the combined light to the combining member 57.

The combining member 57 is configured to reflect the violet light VL incident on the one surface 57a in a direction of the combining member 58 and to transmit light (at least one of the red light RL or the blue light BL), which is emitted from the combining member 56 and incident on the other surface 57b, in the direction of the combining member 58, so as to combine the light (at least one of the red light RL or the blue light BL) emitted from the combining member 56 and the violet light VL and to emit the combined light to the combining member 58.

The combining member 58 is configured to reflect the green light GL of the white light WL incident on the one surface 58a in a direction of the condenser lens 59 and to transmit light (at least one of the red light RL, the blue light BL, or the violet light VL), which is emitted from the combining member 57 and incident on the other surface 58b, in the direction of the condenser lens 59, so as to combine the light (at least one of the red light RL, the blue light BL, or the violet light VL) emitted from the combining member 57 and the green light GL and to emit the combined light to the condenser lens 59. The green light GL emitted from the combining member 58 to the condenser lens 59 has wavelength characteristics, for example, with a central wavelength of 540 nm or more and 560 nm or less and a half-width of 80 nm or more.

The condenser lens 59 condenses the light emitted from the combining member 58 into the light guide 14.

The red light RL emitted from the first light source 51 is transmitted through the combining member 56, the combining member 57, and the combining member 58, and is then introduced into the light guide 14. The blue light BL emitted from the second light source 52 is reflected by the combining member 56, is transmitted through the combining member 57 and the combining member 58, and is then introduced into the light guide 14. The violet light VL emitted from the third light source 53 is reflected by the combining member 57, is transmitted through the combining member 58, and is then introduced into the light guide 14. The green light GL of the white light WL emitted from the fourth light source 54 is reflected by the combining member 58 and is introduced into the light guide 14.

In the light source device 5, an optical path of the light emitted from the fourth light source 54 to the light guide 14 is shorter than an optical path of the light emitted from the first light source 51, the second light source 52, and the third light source 53 to the light guide 14. The fourth light source 54 generates the white light WL by using a light emitting diode and a phosphor having a large light diffusion. The fourth light source 54 is provided at a position closest to the light guide 14 among the four light sources, so that it is possible to easily adjust a positional relationship between a distribution range of the green light GL included in the white light WL and a proximal end surface of the light guide 14.

The light source device 5 is operable in a plurality of modes in which the number of rays of light to be introduced into the light guide 14 of the endoscope 1 varies. The plurality of modes are selected according to the purpose in an endoscope examination, and include a first mode in which the red light RL, the blue light BL, and the green light GL are combined and introduced into the light guide 14, a second mode in which the violet light VL and the green light GL are combined and introduced into the light guide 14, a third mode in which the blue light BL and the violet light VL are combined and introduced into the light guide 14, a fourth mode in which the red light RL, the blue light BL, and the violet light VL are combined and introduced into the light guide 14, and a fifth mode in which the violet light VL, the red light RL, the blue light BL, and the green light GL are combined and introduced into the light guide 14. The third mode and the fourth mode are not essential and may be omitted.

In the first mode, the processor 5P performs a first control of turning on each of the light emitting diodes of the first light source 51, the second light source 52, and the fourth light source 54 to introduce the red light RL, the blue light BL, and the green light GL into the light guide 14.

In the second mode, the processor 5P performs a second control of turning on each of the light emitting diodes of the third light source 53 and the fourth light source 54 to introduce the violet light VL and the green light GL into the light guide 14.

In the third mode, the processor 5P performs a third control of turning on each of the light emitting diodes of the second light source 52 and the third light source 53 to introduce the blue light BL and the violet light VL into the light guide 14.

In the fourth mode, the processor 5P performs a fourth control of turning on each of the light emitting diodes of the first light source 51, the second light source 52, and the third light source 53 to introduce the red light RL, the blue light BL, and the violet light VL into the light guide 14.

In the fifth mode, the processor 5P performs a fifth control of turning on each of the light emitting diodes of the first light source 51, the second light source 52, the third light source 53, and the fourth light source 54 to introduce the red light RL, the blue light BL, the green light GL, and the violet light VL into the light guide 14. In the fifth control, by changing amounts of light generated by the four light sources, it is also possible to further divide the fifth mode into a plurality of modes.

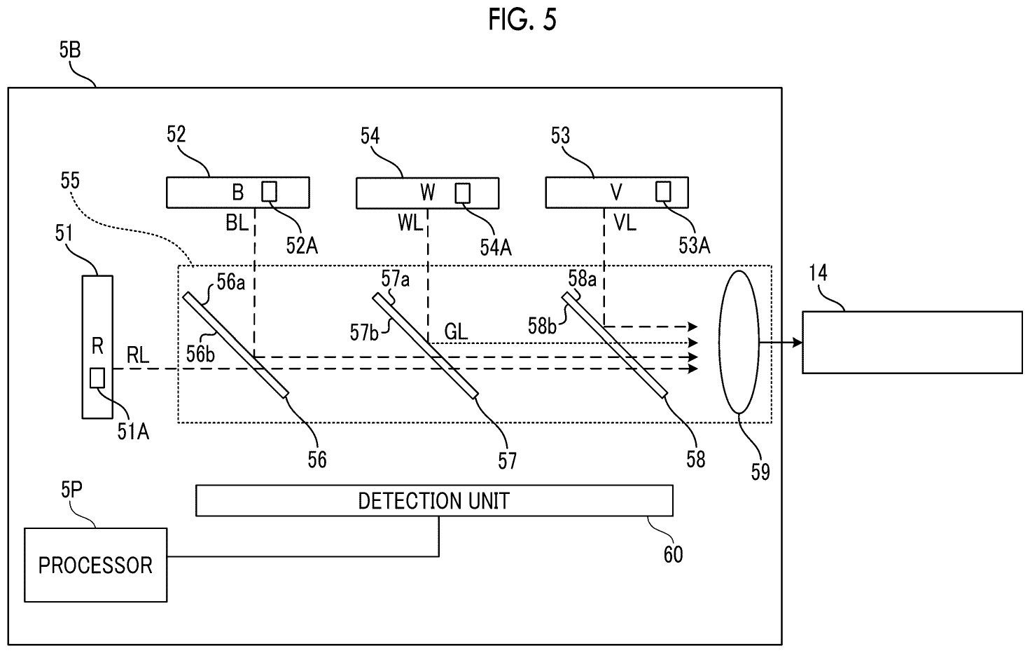

FIG. 5 is a diagram showing a light source device 5B which is a first modification example of the light source device 5.

The light source device 5B has a configuration in which positions of the third light source 53 and the fourth light source 54 are reversed with respect to the light source device 5. In the light source device 5B, the combining member 57 reflects the green light GL of the white light WL incident on the one surface 57a in the direction of the combining member 58. In addition, the combining member 58 transmits the green light GL incident on the other surface 58b in the direction of the condenser lens 59 and reflects the violet light VL incident on the one surface 58a in the direction of the condenser lens 59. The plurality of modes described above can also be implemented by the configuration shown in FIG. 5.

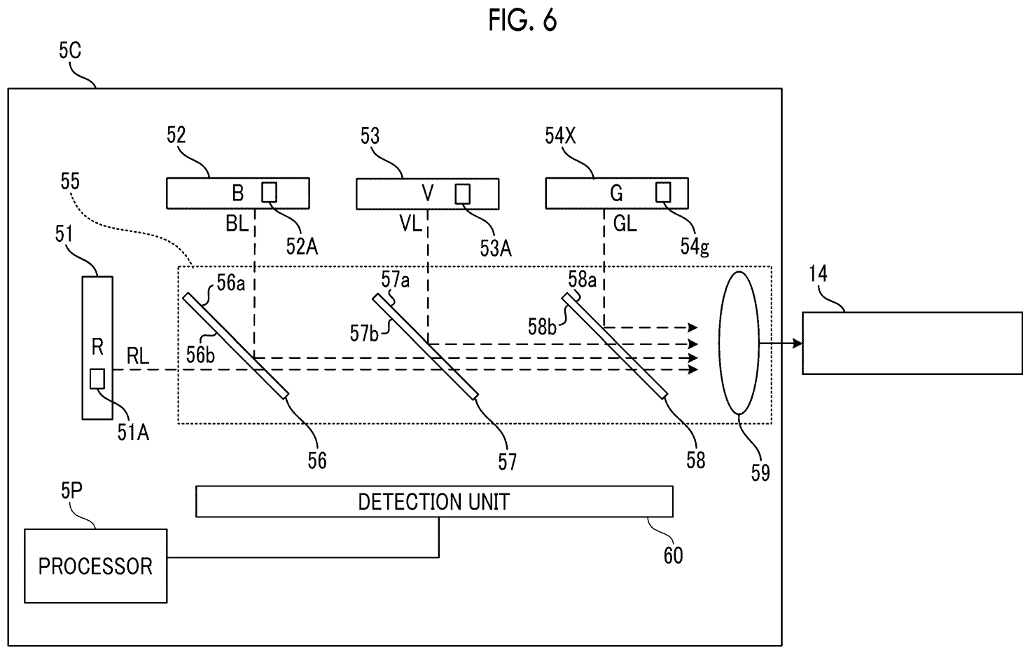

FIG. 6 is a diagram showing a light source device 5C which is a second modification example of the light source device 5.

The light source device 5C has a configuration in which the fourth light source 54 is replaced with a fourth light source 54X with respect to the light source device 5. The fourth light source 54X includes a light emitting diode 54g that generates the green light GL. In the light source device 5C, the combining member 58 reflects the green light GL incident on the one surface 58a in the direction of the condenser lens 59. The plurality of modes described above can also be implemented by the configuration shown in FIG. 6.

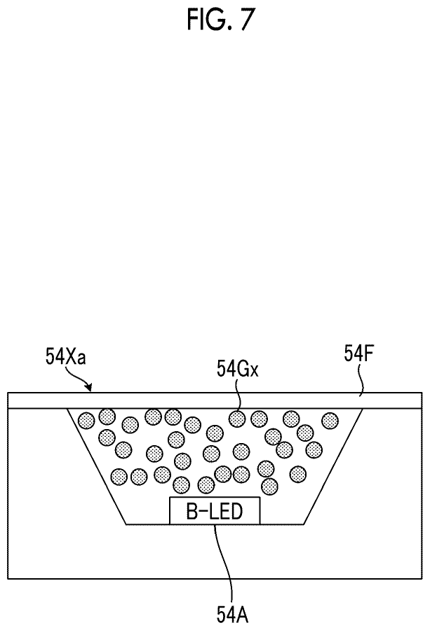

FIG. 7 is a schematic diagram illustrating a configuration of a fourth light source 54Xa which is a modification example of the fourth light source 54X in FIG. 6.

The fourth light source 54Xa includes the light emitting diode 54A that generates blue excitation light B1, a fourth phosphor 54Gx that generates the green light GL by receiving the excitation light B1 generated by the light emitting diode 54A, and an excitation light cut filter 54F that transmits the green light GL and that cuts the excitation light B1. The fourth light source 54Xa need only include a light emitting diode that generates excitation light and a phosphor that generates light including at least the green light GL by receiving the excitation light, and the excitation light is not limited to blue light. For example, in FIG. 7, the light emitting diode 54A may be replaced with a light emitting diode that generates violet light or a light emitting diode that generates ultraviolet light.

The excitation light cut filter 54F need not be provided in the light source itself as shown in FIG. 7. For example, the excitation light cut filter 54F may be provided in an optical path between the combining member 58 and the fourth light source 54X in FIG. 6. In addition, a configuration may be employed in which the combining member 58 transmits a component other than the green light GL, among rays of light incident on the one surface 58a.

In the light source device 5B shown in FIG. 5, the fourth light source 54 can also be replaced with the fourth light source 54X or the fourth light source 54Xa.

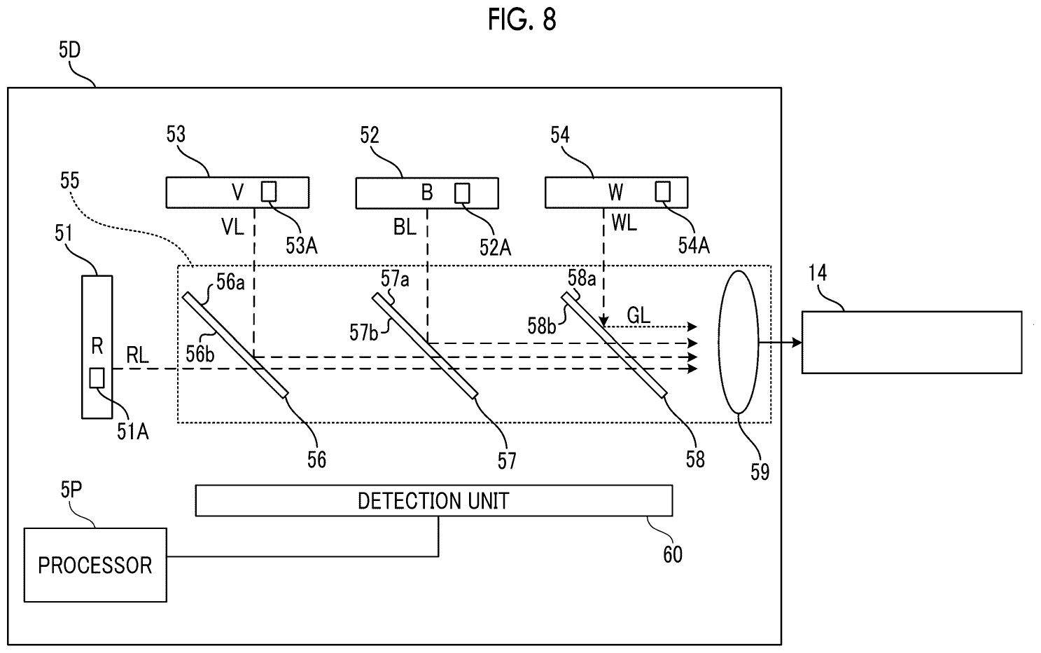

FIG. 8 is a diagram showing a light source device 5D which is a third modification example of the light source device 5.

The light source device 5D has a configuration in which positions of the second light source 52 and the third light source 53 are reversed with respect to the light source device 5. In the light source device 5D, the combining member 56 reflects the violet light VL incident on the one surface 56a in the direction of the combining member 57. In addition, the combining member 57 transmits the violet light VL incident on the other surface 57b in the direction of the combining member 58 and reflects the blue light BL incident on the one surface 57a in the direction of the combining member 58. The plurality of modes described above can also be implemented by the configuration shown in FIG. 8.

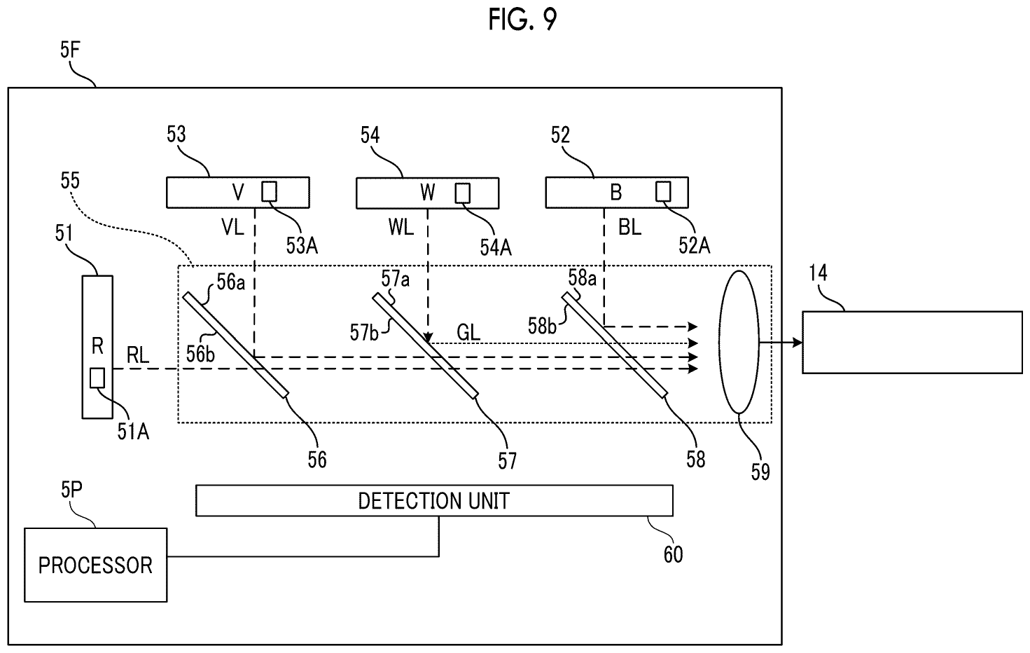

FIG. 9 is a diagram showing a light source device 5F which is a fourth modification example of the light source device 5.

The light source device 5F has a configuration in which positions of the second light source 52 and the fourth light source 54 are reversed with respect to the light source device 5D. In the light source device 5F, the combining member 57 reflects the green light GL of the white light WL incident on the one surface 57a in the direction of the combining member 58. In addition, the combining member 58 transmits the green light GL incident on the other surface 58b in the direction of the condenser lens 59 and reflects the blue light BL incident on the one surface 58a in the direction of the condenser lens 59. The plurality of modes described above can also be implemented by the configuration shown in FIG. 9.

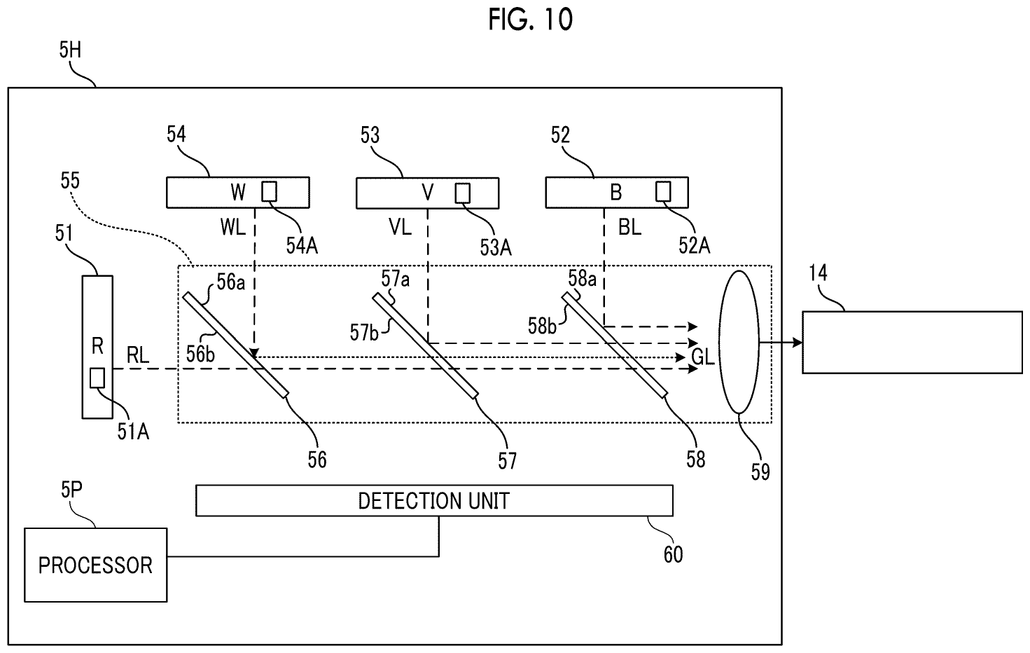

FIG. 10 is a diagram showing a light source device 5H which is a fifth modification example of the light source device 5.

The light source device 5H has a configuration in which the positions of the third light source 53 and the fourth light source 54 are reversed with respect to the light source device 5F. In the light source device 5H, the combining member 56 reflects the green light GL of the white light WL incident on the one surface 56a in the direction of the combining member 57. In addition, the combining member 57 transmits the green light GL incident on the other surface 57b in the direction of the combining member 58 and reflects the violet light VL incident on the one surface 57a in the direction of the combining member 58. The plurality of modes described above can also be implemented by the configuration shown in FIG. 10.

In the light source device 5 and each of the light source devices 5B, 5C, 5D, 5F, and 5H shown so far, positions of the first light source 51 and the second light source 52 may be reversed.

The light to be introduced into the light guide 14 of the endoscope 1 is desired to have bright green light from the viewpoint of increasing brightness of a color image (an image captured in a state in which the subject is irradiated with the red light RL, the green light GL, and the blue light BL) captured by the endoscope 1. In addition, it is desired that green light and violet light are bright from the viewpoint of emphasizing and clearly displaying capillaries of a mucous membrane surface layer, patterns thereof, and the like during special light observation.

According to the configurations of the light source device 5 and the light source devices 5B and 5C, among rays of light to be introduced into the light guide 14, the green light GL and the violet light VL have less attenuation due to transmission or reflection through the optical member 55 as compared with the other two colors of light. Therefore, it is possible to enhance quality of the captured image captured by the endoscope 1.

In particular, according to the configurations of the light source device 5 and the light source device 5C, among rays of light to be introduced into the light guide 14, the green light GL has less attenuation due to transmission or reflection through the optical member 55 as compared with the other three colors of light. Therefore, it is possible to enhance the quality of the captured image captured by the endoscope 1.

Next, a specific configuration example of the detection unit 60 and a specific processing example of the processor 5P will be described.

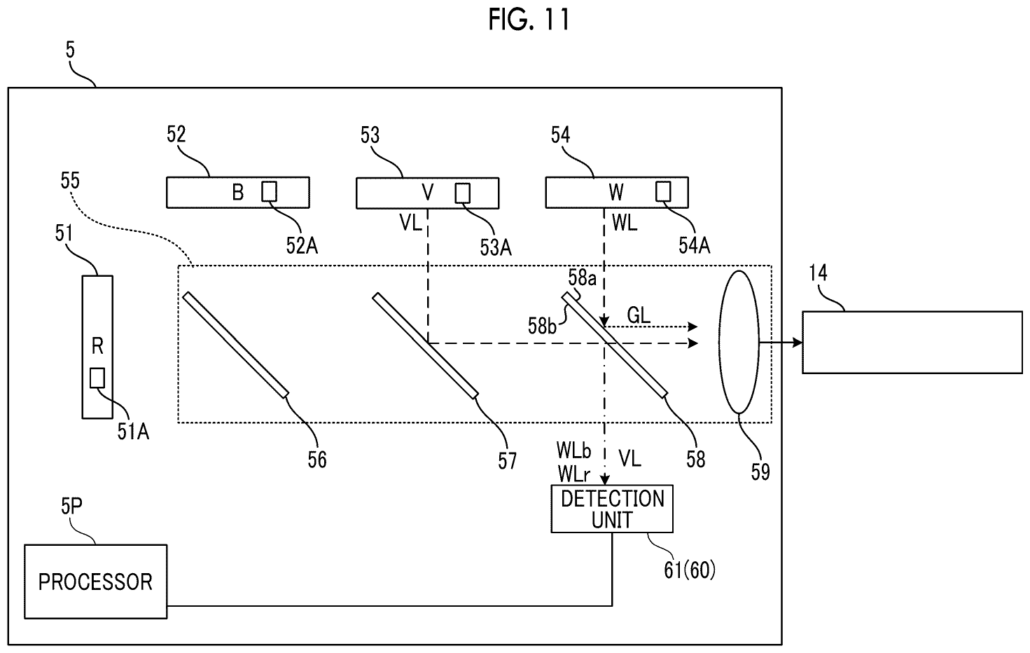

FIG. 11 is a schematic diagram showing a configuration example of the detection unit 60 in the light source device 5. FIG. 11 shows a state in which the light source device 5 operates in the second mode.

In the example of FIG. 11, one detection unit 61 including a light-receiving element such as a photodiode or a photoresistor is provided as the detection unit 60. The detection unit 61 is provided on the other side (a lower side in the drawing) in the direction perpendicular to the optical axis direction of the condenser lens 59 with respect to the combining member 58.

In the light source device 5 shown in FIG. 11, the combining member 58 has a function of reflecting a part of the violet light VL incident on the other surface 58b in a direction of the detection unit 61, of transmitting blue light (hereinafter, referred to as blue light WLb) of the white light WL incident on the one surface 58a in the direction of the detection unit 61, and of transmitting red light (hereinafter, referred to as red light WLr) of the white light WL incident on the one surface 58a in the direction of the detection unit 61.

The detection unit 61 is configured to be capable of detecting the violet light VL reflected by the other surface 58b of the combining member 58, and the blue light WLb and the red light WLr incident on the one surface 58a of the combining member 58 and transmitted through the combining member 58.

In the light source device 5 shown in FIG. 11, the combining member 58 may transmit only any one of the blue light WLb or the red light WLr in the direction of the detection unit 61. In this case, the detection unit 61 can detect the violet light VL reflected by the other surface 58b of the combining member 58, and the blue light WLb or the red light WLr incident on the one surface 58a of the combining member 58 and transmitted through the combining member 58.

In the light source device 5 having the configuration shown in FIG. 11, in the second mode, the processor 5P controls the amount of the violet light VL generated by the third light source 53 and the amount of the white light WL generated by the fourth light source 54, based on information on the violet light VL detected by the detection unit 61 and information on at least one of the blue light WLb or the red light WLr detected by the detection unit 61.

The information on the light detected by the detection unit 60 is, for example, a spectrum indicating an intensity for each wavelength. The control performed by the processor 5P on the amounts of light of the plurality of light sources includes a control of setting the amount of light of each of the plurality of light sources to a desired value and a control of setting a light amount ratio of the plurality of light sources to a desired value.

In the light source device 5 having the configuration shown in FIG. 11, the processor 5P determines a ratio between an amount of light of the third light source 53 and an amount of light of the fourth light source 54, for example, from a relationship between a peak value of an intensity of the violet light VL and a peak value of an intensity of the blue light WLb.

Alternatively, the processor 5P determines the ratio between the amount of light of the third light source 53 and the amount of light of the fourth light source 54 from a relationship between the peak value of the intensity of the violet light VL and a peak value of an intensity of the red light WLr.

Alternatively, the processor 5P derives a peak value of an intensity of the green light GL from the peak value of the intensity of the blue light WLb and the peak value of the intensity of the red light WLr and determines the ratio between the amount of light of the third light source 53 and the amount of light of the fourth light source 54 from a relationship between the derived peak value of the intensity of the green light GL and the peak value of the intensity of the violet light VL. Here, the peak value of the intensity of the detected light is used, but an integrated value obtained from the spectrum may be used instead of the peak value.

In FIG. 11, the combining member 58 may be configured to transmit a part of the green light GL incident on the one surface 58a in the direction of the detection unit 61. In this case, the processor 5P can also determine the ratio between the amount of light of the third light source 53 and the amount of light of the fourth light source 54, for example, from the relationship between the peak value of the intensity of the violet light VL and the peak value of the intensity of the green light GL.

In the optical member 55, the green light GL is introduced into the light guide 14 through only one reflection. Therefore, even with a configuration in which a part of the green light GL is detected by the detection unit 61, it is possible to sufficiently increase the amount of the green light GL to be introduced into the light guide 14, and it is possible to enhance the quality of the captured image captured by the endoscope 1.

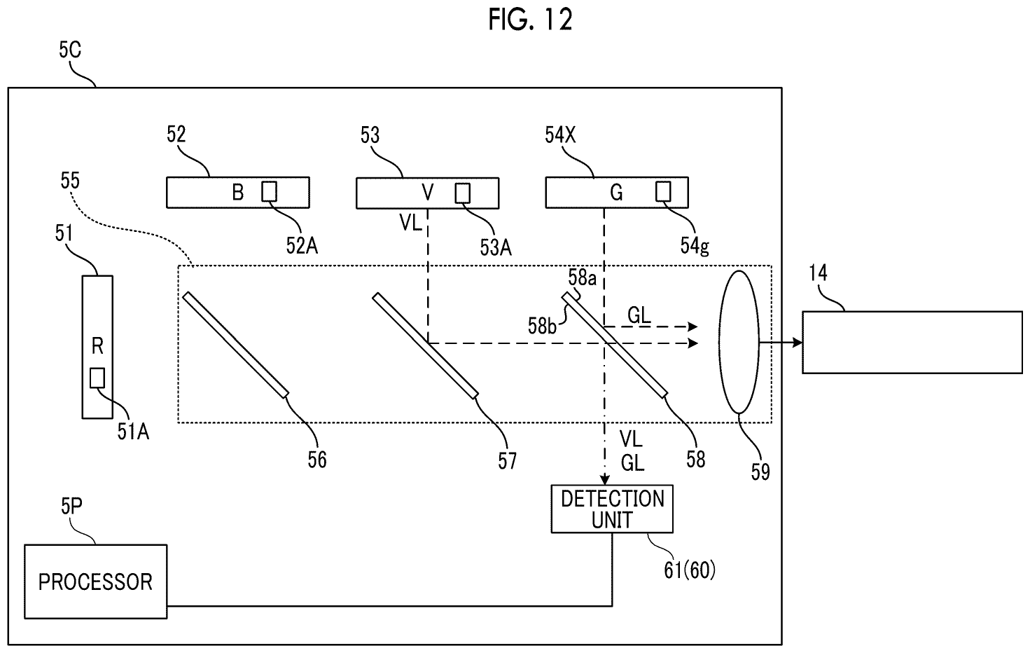

FIG. 12 is a schematic diagram showing a configuration example of the detection unit 60 in the light source device 5C. FIG. 12 shows a state in which the light source device 5C operates in the second mode.

The light source device 5C shown in FIG. 12 is different from the light source device 5 shown in FIG. 11 in that the fourth light source 54 is replaced with the fourth light source 54X, and in that the combining member 58 transmits a part of the green light GL incident on the one surface 58a in the direction of the detection unit 61.

In the light source device 5C having the configuration shown in FIG. 12, in the second mode, the processor 5P controls the amount of the violet light VL generated by the third light source 53 and the amount of the green light GL generated by the fourth light source 54X, based on the information on the violet light VL detected by the detection unit 61 and information on the green light GL detected by the detection unit 61. The processor 5P determines a ratio between the amount of light of the third light source 53 and the amount of light of the fourth light source 54X, for example, from the relationship between the peak value of the intensity of the violet light VL and the peak value of the intensity of the green light GL.

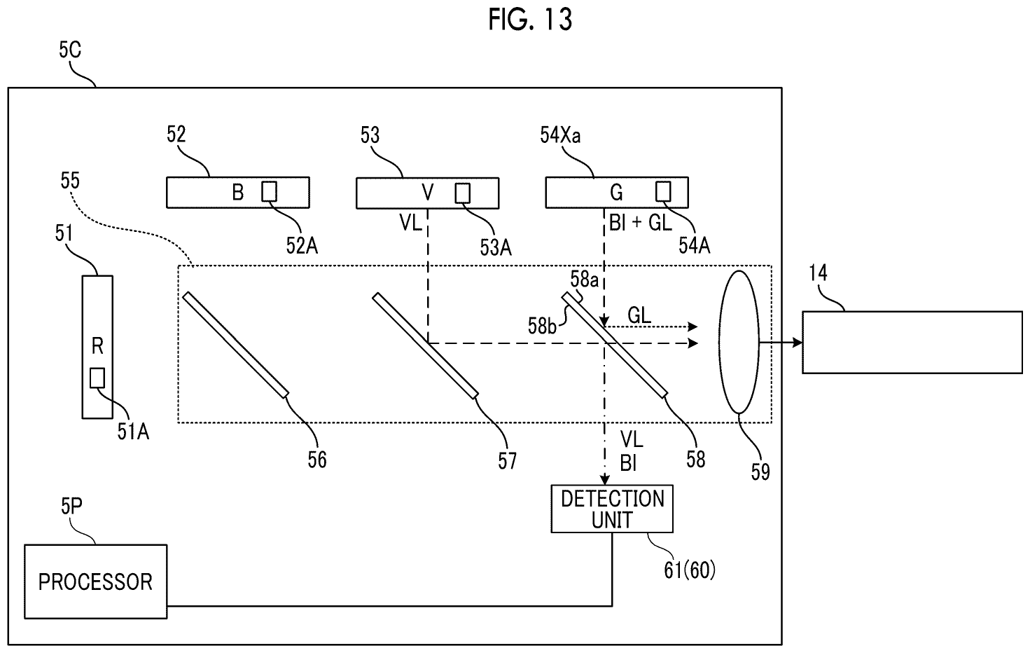

FIG. 13 is a schematic diagram showing a configuration example of the detection unit 60 in the light source device 5C. FIG. 13 shows a state in which the light source device 5C operates in the second mode.

The light source device 5C shown in FIG. 13 is different from the light source device 5 shown in FIG. 11 in that the fourth light source 54 is replaced with the fourth light source 54Xa having a configuration in which the excitation light cut filter 54F is removed, and in that the combining member 58 transmits the excitation light B1 incident on the one surface 58a in the direction of the detection unit 61.

In the light source device 5C having the configuration shown in FIG. 13, in the second mode, the processor 5P controls the amount of the violet light VL generated by the third light source 53 and the amount of the green light GL generated by the fourth light source 54Xa (the amount of light emitted from the light emitting diode 54A), based on the information on the violet light VL detected by the detection unit 61 and information on the excitation light B1 detected by the detection unit 61. The processor 5P determines a ratio between the amount of light of the third light source 53 and the amount of light of the fourth light source 54Xa, for example, from a relationship between the peak value of the intensity of the violet light VL and a peak value of an intensity of the excitation light B1.

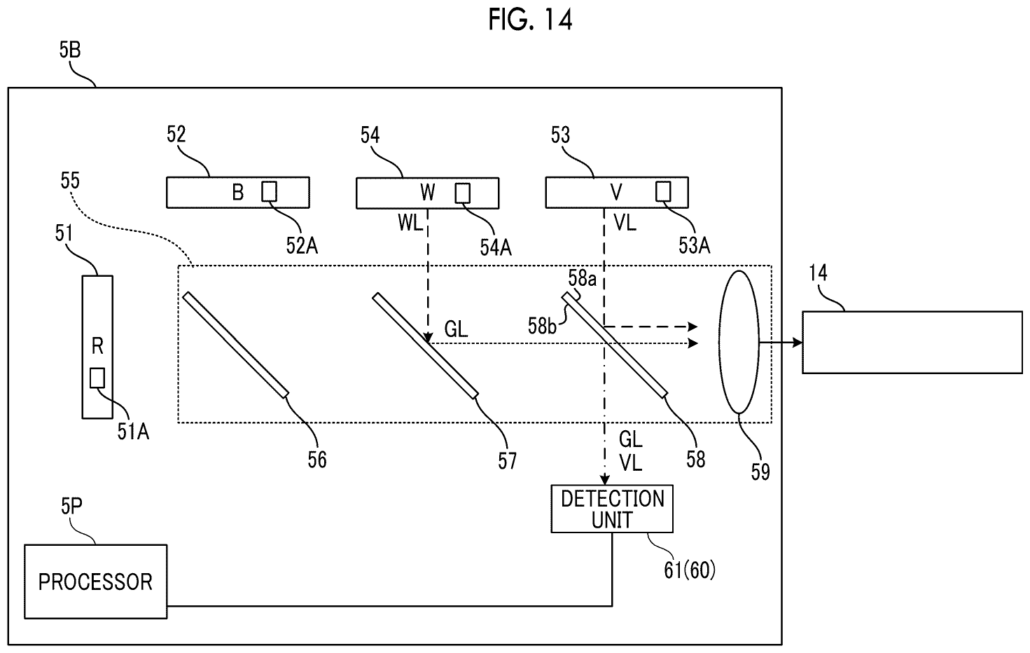

FIG. 14 is a schematic diagram showing a configuration example of the detection unit 60 in the light source device 5B. FIG. 14 shows a state in which the light source device 5B operates in the second mode.

In the example of FIG. 14, one detection unit 61 including the light-receiving element such as a photodiode or a photoresistor is provided as the detection unit 60. The detection unit 61 is provided on the other side (the lower side in the drawing) in the direction perpendicular to the optical axis direction of the condenser lens 59 with respect to the combining member 58.

In the light source device 5B shown in FIG. 14, the combining member 58 has a function of transmitting a part of the violet light VL incident on the one surface 58a in the direction of the detection unit 61 and of transmitting a part of the green light GL incident on the other surface 58b in the direction of the detection unit 61. The detection unit 61 shown in FIG. 14 is configured to be capable of detecting the violet light VL transmitted through the one surface 58a of the combining member 58, and the green light GL incident on the other surface 58b of the combining member 58 and reflected by the combining member 58.

The control performed by the processor 5P on the amount of light of the light source in the second mode of the light source device 5B having the configuration shown in FIG. 14 is the same as that of the processor 5P shown in FIG. 12. In the light source device 5B shown in FIG. 14, the fourth light source 54 may be changed to the fourth light source 54X or the fourth light source 54Xa.

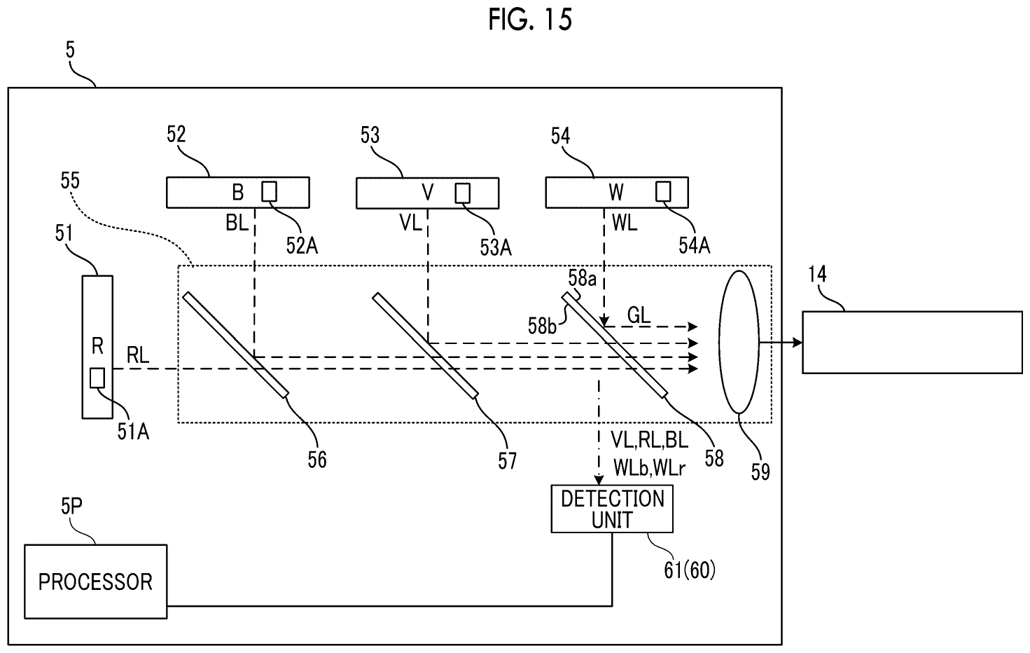

FIG. 15 is a diagram showing a modification example of the light source device 5 shown in FIG. 11. FIG. 15 shows a state in which the light source device 5 operates in the fifth mode.

The light source device 5 shown in FIG. 15 is different from the light source device 5 shown in FIG. 11 in that the combining member 58 reflects a part of the red light RL and a part of the blue light BL incident on the other surface 58b in the direction of the detection unit 61. The detection unit 61 shown in FIG. 15 is configured to capable of detecting the violet light VL, the red light RL, and the blue light BL reflected by the other surface 58b of the combining member 58, and the blue light WLb and the red light WLr incident on the one surface 58a of the combining member 58 and transmitted through the combining member 58.

In the light source device 5 having the configuration shown in FIG. 15, in the fifth mode, the processor 5P controls the amount of the red light RL generated by the first light source 51, the amount of the blue light BL generated by the second light source 52, the amount of the violet light VL generated by the third light source 53, and the amount of the white light WL generated by the fourth light source 54, based on information on each of the violet light VL, the red light RL, the blue light BL, the blue light WLb, and the red light WLr detected by the detection unit 61.

For example, the processor 5P derives the amount of the green light GL based on the peak value of the intensity of the red light WLr. The processor 5P derives the amount of the blue light BL from a relationship between a peak value of an intensity of the detected blue light and the peak value of the intensity of the red light WLr. The processor 5P derives the amount of the violet light VL from the peak value of the intensity of the detected violet light VL. The processor 5P derives the amount of the red light RL from the peak value of the intensity of the detected red light WLr. The processor 5P controls the amount of the red light RL generated by the first light source 51, the amount of the blue light BL generated by the second light source 52, the amount of the violet light VL generated by the third light source 53, and the amount of the white light WL generated by the fourth light source 54, based on the amount of each color of light derived in this way.

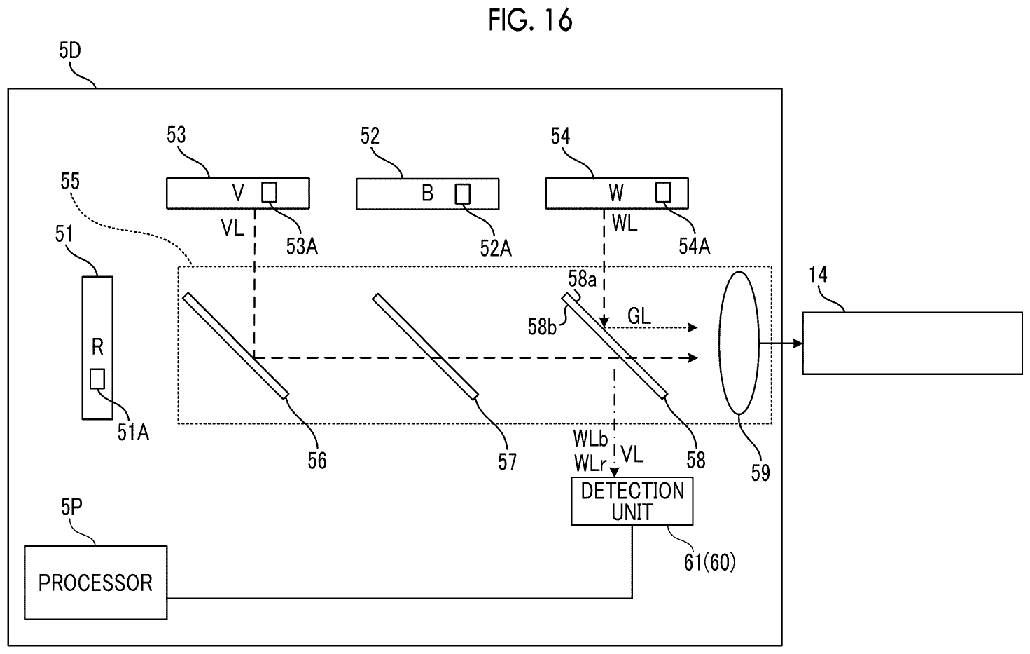

FIG. 16 is a schematic diagram showing a configuration example of the detection unit 60 in the light source device 5D. FIG. 16 shows a state in which the light source device 5D operates in the second mode.

The light source device 5D shown in FIG. 16 is different from the light source device 5 shown in FIG. 11 in that the positions of the second light source 52 and the third light source 53 are reversed. The control performed by the processor 5P on the amount of light of the light source in the second mode of the light source device 5D having the configuration shown in FIG. 16 is the same as that of the processor 5P shown in FIG. 11.

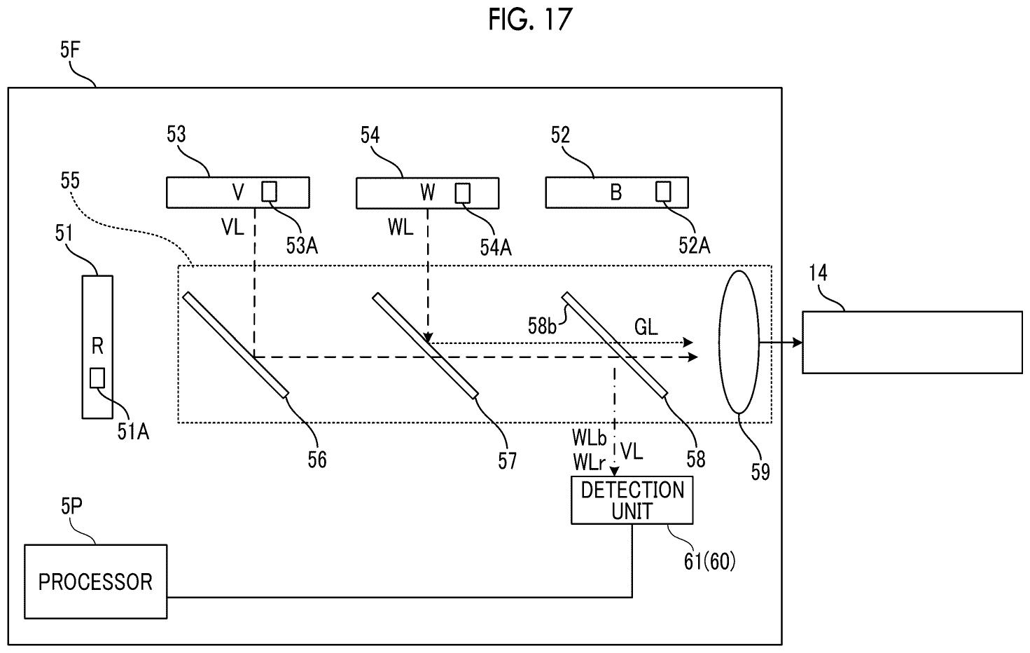

FIG. 17 is a schematic diagram showing a configuration example of the detection unit 60 in the light source device 5F. FIG. 17 shows a state in which the light source device 5F operates in the second mode.

The light source device 5F shown in FIG. 17 is different from the light source device 5D shown in FIG. 16 in that the positions of the second light source 52 and the fourth light source 54 are reversed, and in that the combining member 58 is configured to reflect a part of the violet light VL incident on the other surface 58b in the direction of the detection unit 61 and to reflect a part of the green light GL incident on the other surface 58b in the direction of the detection unit 61. The control performed by the processor 5P on the amount of light of the light source in the second mode of the light source device 5F having the configuration shown in FIG. 17 is the same as that of the processor 5P shown in FIG. 14.

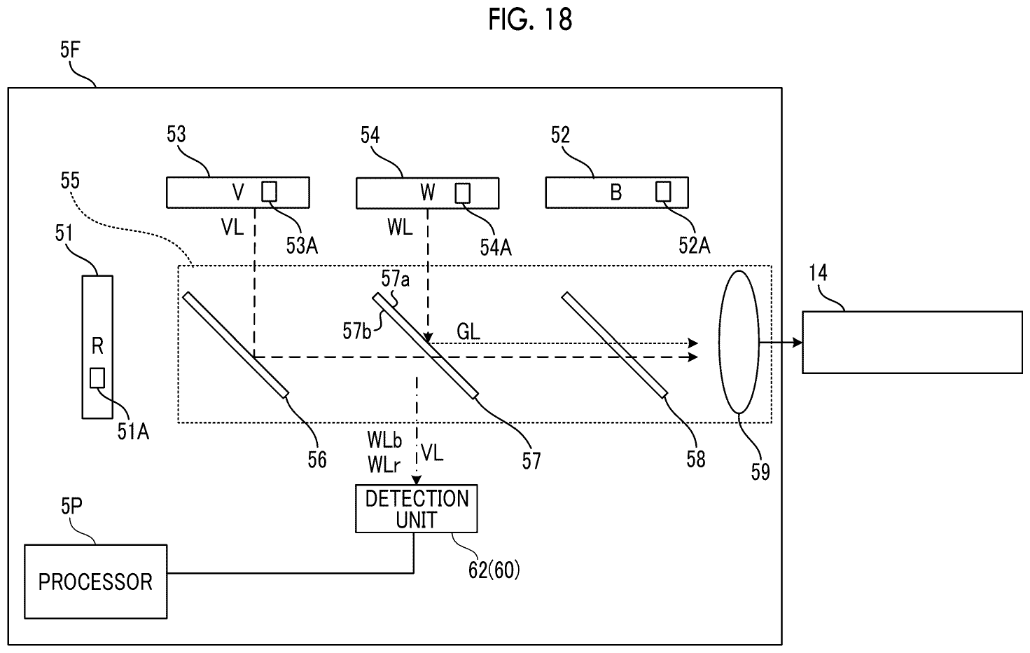

FIG. 18 is a schematic diagram showing a configuration example of the detection unit 60 in the light source device 5F. FIG. 18 shows a state in which the light source device 5F operates in the second mode.

In the light source device 5F shown in FIG. 18, one detection unit 62 including a light-receiving element such as a photodiode or a photoresistor is provided as the detection unit 60. The detection unit 62 is provided on the other side (the lower side in the drawing) in the direction perpendicular to the optical axis direction of the condenser lens 59 with respect to the combining member 57.

In the light source device 5F shown in FIG. 18, the combining member 57 has a function of reflecting a part of the violet light VL incident on the other surface 57b in a direction of the detection unit 62 and of transmitting at least one of the blue light WLb or the red light WLr of the white light WL incident on the one surface 57a in the direction of the detection unit 62. The detection unit 62 is configured to be capable of detecting the violet light VL reflected by the other surface 57b of the combining member 57, and the blue light WLb and the red light WLr incident on the one surface 57a of the combining member 57 and transmitted through the combining member 57. The combining member 57 may be configured to transmit a part of the green light GL incident on the one surface 57a in the direction of the detection unit 62.

In the light source device 5F having the configuration shown in FIG. 18, in the second mode, the processor 5P controls the amount of the violet light VL generated by the third light source 53 and the amount of the white light WL generated by the fourth light source 54, based on information on the violet light VL detected by the detection unit 62 and information on at least one of the blue light WLb or the red light WLr detected by the detection unit 62 (or information on the green light GL detected by the detection unit 62).

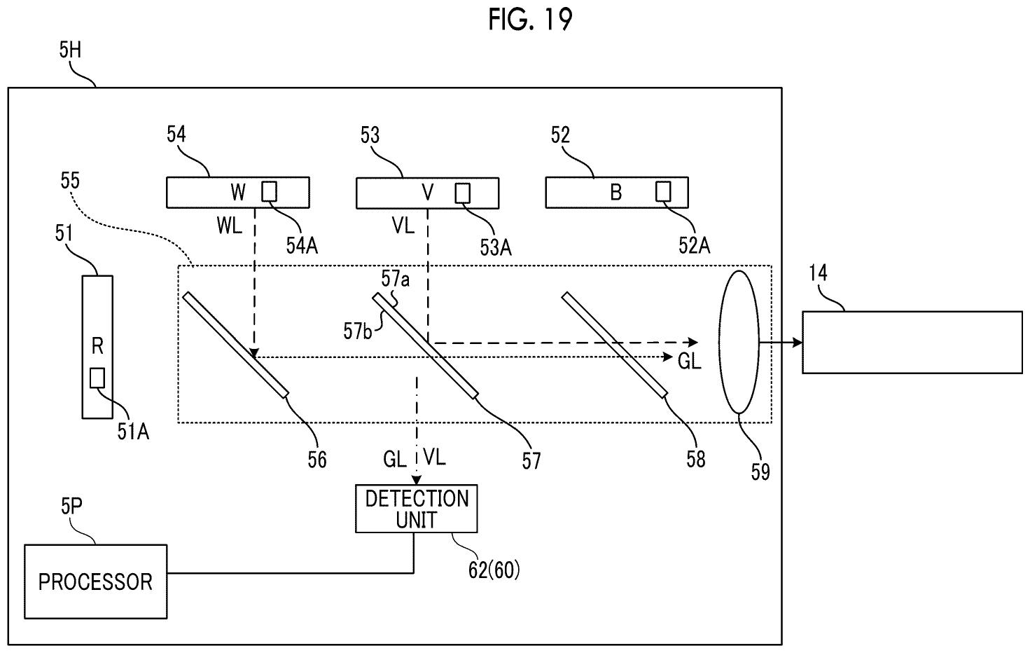

FIG. 19 is a schematic diagram showing a configuration example of the detection unit 60 in the light source device 5H. FIG. 19 shows a state in which the light source device 5H operates in the second mode.

The light source device 5H shown in FIG. 19 is different from the light source device 5F shown in FIG. 18 in that the positions of the third light source 53 and the fourth light source 54 are reversed, and in that the combining member 57 is configured to reflect a part of the green light GL incident on the other surface 57b in the direction of the detection unit 62 and to transmit a part of the violet light VL incident on the one surface 57a in the direction of the detection unit 62.

The control performed by the processor 5P on the amount of light of the light source in the second mode of the light source device 5H having the configuration shown in FIG. 19 is the same as that of the processor 5P shown in FIG. 14.

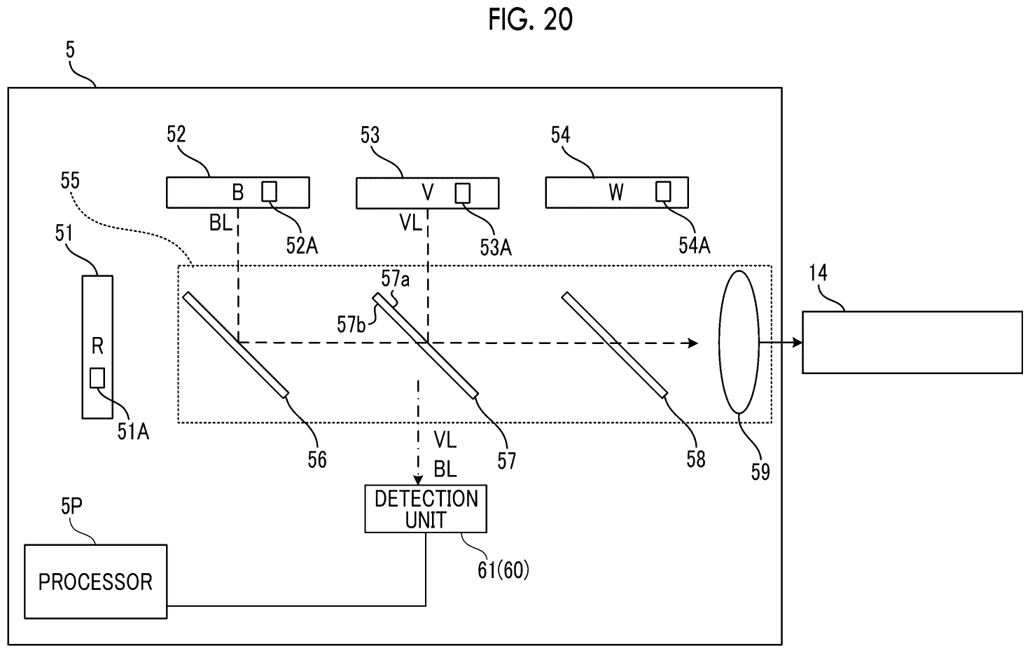

FIG. 20 is a schematic diagram showing a configuration example of the detection unit 60 in the light source device 5. FIG. 20 shows a state in which the light source device 5 operates in the third mode.

In the example of FIG. 20, one detection unit 61 including the light-receiving element such as a photodiode or a photoresistor is provided as the detection unit 60. The detection unit 61 is provided on the other side (the lower side in the drawing) in the direction perpendicular to the optical axis direction of the condenser lens 59 with respect to the combining member 57.

In the light source device 5 shown in FIG. 20, the combining member 57 has a function of reflecting a part of the blue light BL incident on the other surface 57b in the direction of the detection unit 61 and of transmitting a part of the violet light VL incident on the one surface 57a in the direction of the detection unit 61. The detection unit 61 is configured to be capable of detecting the blue light BL reflected by the other surface 57b of the combining member 57, and the violet light VL incident on the one surface 57a of the combining member 57 and transmitted through the combining member 57.

In the light source device 5 having the configuration shown in FIG. 20, in the third mode, the processor 5P controls the amount of the blue light BL generated by the second light source 52 and the amount of the violet light VL generated by the third light source 53, based on the information on the violet light VL detected by the detection unit 61, and the information on the blue light BL detected by the detection unit 61.

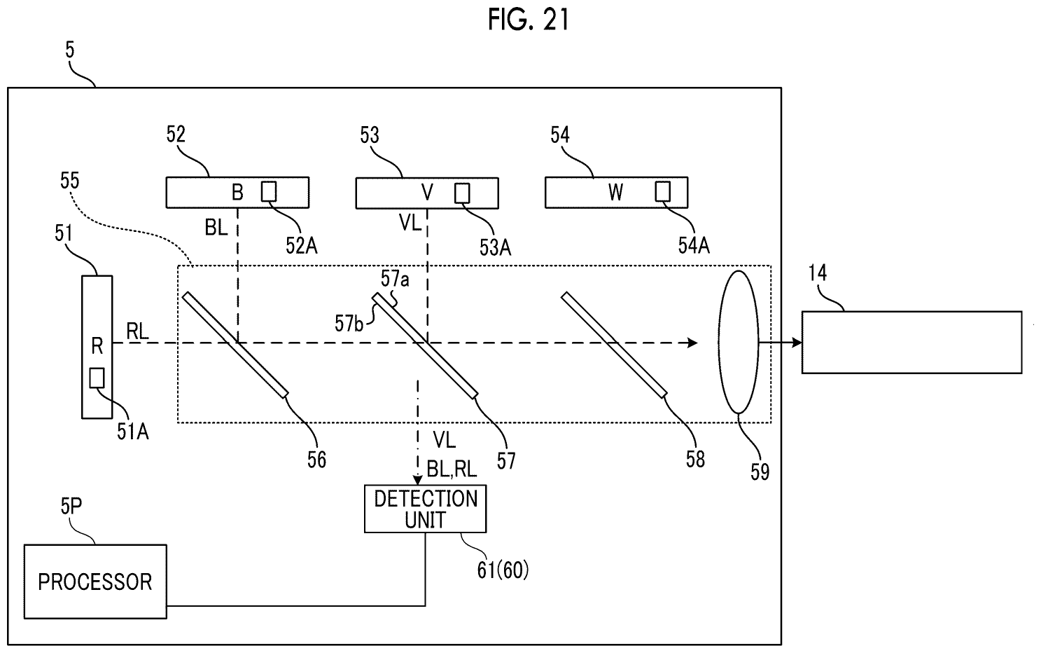

FIG. 21 is a schematic diagram showing a configuration example of the detection unit 60 in the light source device 5. FIG. 21 shows a state in which the light source device 5 operates in the fourth mode.

The light source device 5 shown in FIG. 21 is different from the light source device 5 shown in FIG. 20 in that the combining member 57 is configured to reflect a part of the red light RL incident on the other surface 57b in the direction of the detection unit 61. The detection unit 61 is configured to be capable of detecting a part of the blue light BL and a part of the red light RL, which are reflected by the other surface 57b of the combining member 57, and a part of the violet light VL incident on the one surface 57a of the combining member 57 and transmitted through the combining member 57.

In the light source device 5 having the configuration shown in FIG. 21, in the fourth mode, the processor 5P controls the amount of the red light RL generated by the first light source 51, the amount of the blue light BL generated by the second light source 52, and the amount of the violet light VL generated by the third light source 53, based on the information on the violet light VL detected by the detection unit 61, the information on the blue light BL detected by the detection unit 61, and the information on the red light RL detected by the detection unit 61.

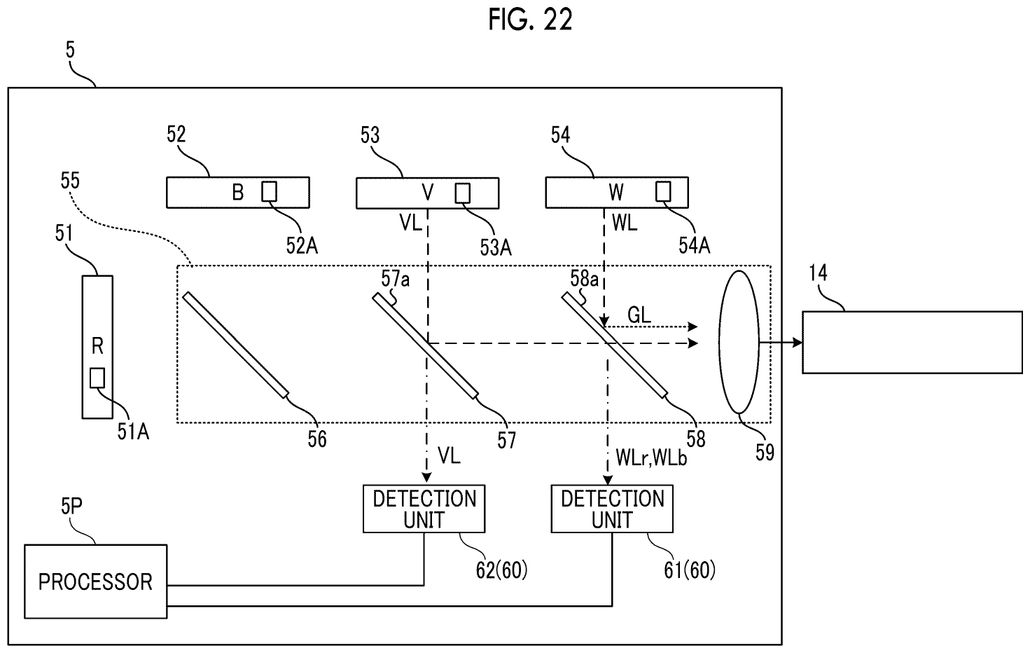

FIG. 22 is a schematic diagram showing a configuration example of the detection unit 60 in the light source device 5. FIG. 22 shows a state in which the light source device 5 operates in the second mode.

In the example of FIG. 22, the detection unit 61 and the detection unit 62, each including the light-receiving element such as a photodiode or a photoresistor, are provided as the detection units 60. The detection unit 61 is provided on the other side (the lower side in the drawing) in the direction perpendicular to the optical axis direction of the condenser lens 59 with respect to the combining member 58. The detection unit 62 is provided on the other side (the lower side in the drawing) in the direction perpendicular to the optical axis direction of the condenser lens 59 with respect to the combining member 57.

In the light source device 5 shown in FIG. 22, the combining member 57 has a function of transmitting a part of the violet light VL incident on the one surface 57a in the direction of the detection unit 62. The combining member 58 has a function of transmitting the red light WLr and the blue light WLb of the white light WL incident on the one surface 58a in the direction of the detection unit 61. In FIG. 22, the combining member 58 may be configured to transmit a part of the green light GL incident on the one surface 58a in the direction of the detection unit 61.

In the light source device 5 having the configuration shown in FIG. 22, in the second mode, the processor 5P controls the amount of the violet light VL generated by the third light source 53 and the amount of the white light WL generated by the fourth light source 54, based on the information on the violet light VL detected by the detection unit 62, and the information on the blue light WLb and the red light WLr detected by the detection unit 61 (or the information on the green light GL detected by the detection unit 61).

For example, the processor 5P determines the amount of light of the third light source 53 and the amount of light of the fourth light source 54 from the relationship between the peak value of the intensity of the violet light VL and the peak value of the intensity of the blue light WLb.

Alternatively, the processor 5P determines the amount of light of the third light source 53 and the amount of light of the fourth light source 54 from the relationship between the peak value of the intensity of the violet light VL and the peak value of the intensity of the red light WLr.

Alternatively, the processor 5P derives the peak value of the intensity of the green light GL from the peak value of the intensity of the blue light WLb and the peak value of the intensity of the red light WLr, determines the amount of light of the fourth light source 54 from the derived peak value of the intensity of the green light GL, and determines the amount of light of the third light source 53 from the peak value of the intensity of the violet light VL.

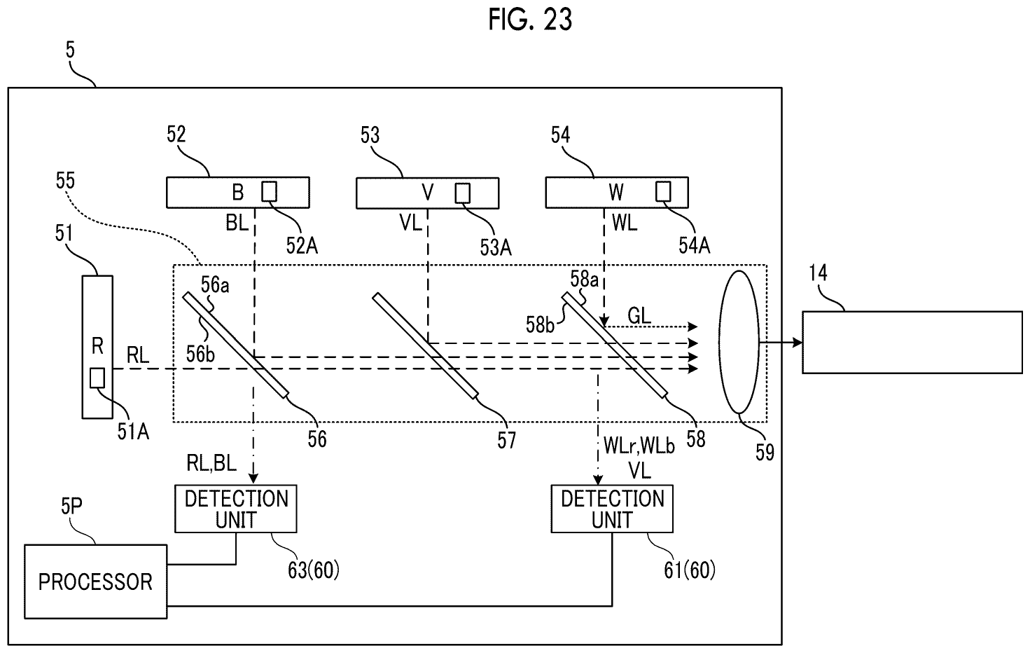

FIG. 23 is a schematic diagram showing a configuration example of the detection unit 60 in the light source device 5. FIG. 23 shows a state in which the light source device 5 operates in the fifth mode.

In the example of FIG. 23, the detection unit 61 and a detection unit 63, each including a light-receiving element such as a photodiode or a photoresistor, are provided as the detection units 60. The detection unit 61 is provided on the other side (the lower side in the drawing) in the direction perpendicular to the optical axis direction of the condenser lens 59 with respect to the combining member 58. The detection unit 63 is provided on the other side (the lower side in the drawing) in the direction perpendicular to the optical axis direction of the condenser lens 59 with respect to the combining member 56.

In the light source device 5 shown in FIG. 23, the combining member 56 has a function of transmitting a part of the blue light BL incident on the one surface 56a in a direction of the detection unit 63 and of reflecting a part of the red light RL incident on the other surface 56b in the direction of the detection unit 63. The combining member 58 has a function of transmitting the red light WLr and the blue light WLb of the white light WL incident on the one surface 58a in the direction of the detection unit 61 and of reflecting a part of the violet light VL incident on the other surface 58b in the direction of the detection unit 61.

In the light source device 5 having the configuration shown in FIG. 23, in the fifth mode, the processor 5P controls the amount of the red light RL generated by the first light source 51, the amount of the blue light BL generated by the second light source 52, the amount of the violet light VL generated by the third light source 53, and the amount of the white light WL generated by the fourth light source 54, based on the information on the violet light VL, the blue light WLb, and the red light WLr detected by the detection unit 61, and the information on the red light RL and the blue light BL detected by the detection unit 63.

For example, the processor 5P determines a ratio between the amount of light of the first light source 51 and the amount of light of the second light source 52 from the peak value of the intensity of the blue light BL and the peak value of the intensity of the red light RL. In addition, the processor 5P derives the peak value of the intensity of the green light GL from the peak value of the intensity of the blue light WLb and the peak value of the intensity of the red light WLr and determines the ratio between the amount of light of the third light source 53 and the amount of light of the fourth light source 54 from the derived peak value of the intensity of the green light GL and the peak value of the intensity of the violet light VL.

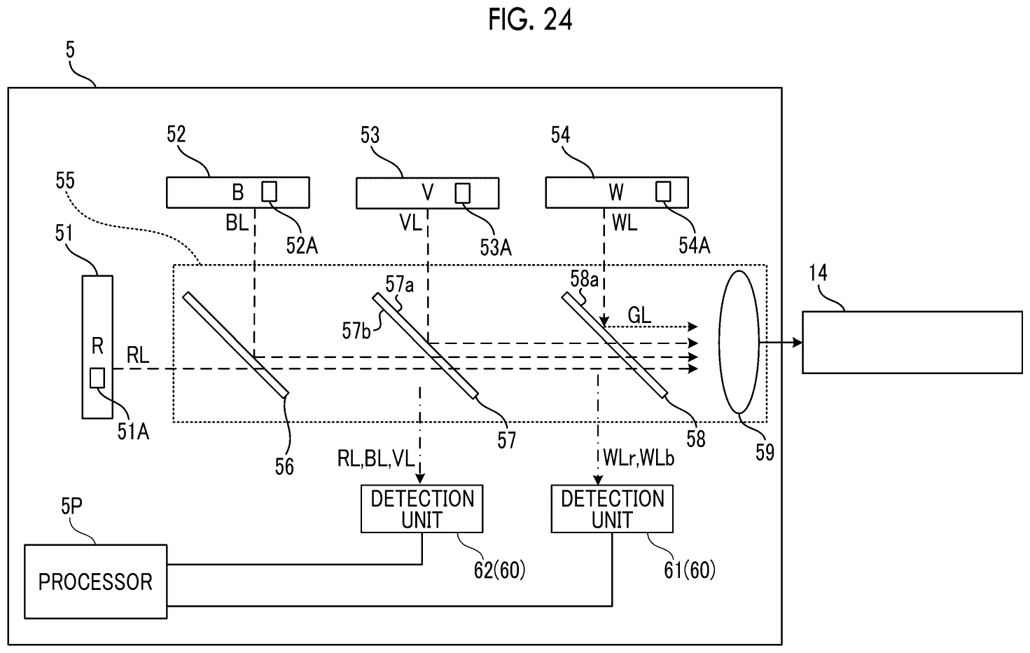

FIG. 24 is a schematic diagram showing a configuration example of the detection unit 60 in the light source device 5. FIG. 24 shows a state in which the light source device 5 operates in the fifth mode.

In the example of FIG. 24, the detection unit 61 and the detection unit 62, each including the light-receiving element such as a photodiode or a photoresistor, are provided as the detection units 60. The detection unit 61 is provided on the other side (the lower side in the drawing) in the direction perpendicular to the optical axis direction of the condenser lens 59 with respect to the combining member 58. The detection unit 62 is provided on the other side (the lower side in the drawing) in the direction perpendicular to the optical axis direction of the condenser lens 59 with respect to the combining member 57.

In the light source device 5 shown in FIG. 24, the combining member 57 has a function of transmitting a part of the violet light VL incident on the one surface 57a in the direction of the detection unit 62, of reflecting a part of the red light RL incident on the other surface 57b in the direction of the detection unit 62, and of reflecting a part of the blue light BL incident on the other surface 57b in the direction of the detection unit 62. The combining member 58 has a function of transmitting the red light WLr and the blue light WLb of the white light WL incident on the one surface 58a in the direction of the detection unit 61.

In the light source device 5 having the configuration shown in FIG. 24, in the fifth mode, the processor 5P controls the amount of the red light RL generated by the first light source 51, the amount of the blue light BL generated by the second light source 52, the amount of the violet light VL generated by the third light source 53, and the amount of the white light WL generated by the fourth light source 54, based on the information on the blue light WLb and the red light WLr detected by the detection unit 61, and the information on the violet light VL, the red light RL, and the blue light BL detected by the detection unit 62.

For example, the processor 5P determines the light amount ratio between the first light source 51, the second light source 52, and the third light source 53 from the peak value of the intensity of the blue light BL, the peak value of the intensity of the red light RL, and the peak value of the intensity of the violet light VL. In addition, the processor 5P derives the peak value of the intensity of the green light GL from the peak value of the intensity of the blue light WLb and the peak value of the intensity of the red light WLr and determines the amount of light of the fourth light source 54 from a relationship between the derived peak value and any one of the peak value of the intensity of the blue light BL, the peak value of the intensity of the red light RL, or the peak value of the intensity of the violet light VL.

The amount of light of each light source may be determined by determining the characteristics of the combining members such that the detection unit 61 detects a part of the red light WLr, a part of the blue light WLb, and a part of the violet light VL, and the detection unit 62 detects a part of the red light RL and a part of the blue light BL.

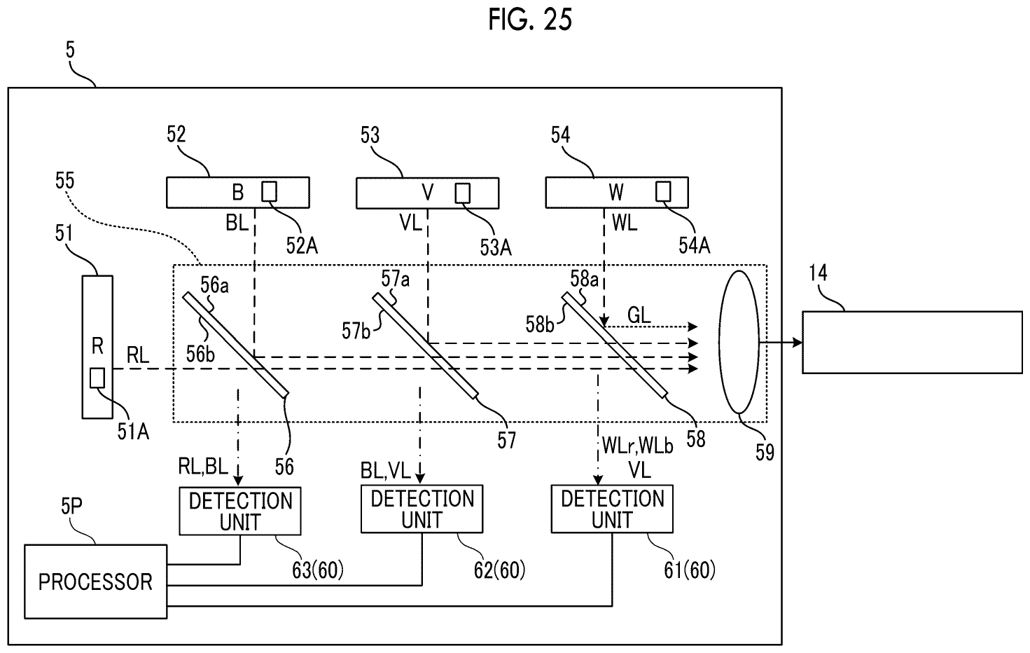

FIG. 25 is a schematic diagram showing a configuration example of the detection unit 60 in the light source device 5. FIG. 25 shows a state in which the light source device 5 operates in the fifth mode.

In the example of FIG. 25, the detection unit 61, the detection unit 62, and the detection unit 63, each including the light-receiving element such as a photodiode or a photoresistor, are provided as the detection units 60. The detection unit 61 is provided on the other side (the lower side in the drawing) in the direction perpendicular to the optical axis direction of the condenser lens 59 with respect to the combining member 58. The detection unit 62 is provided on the other side (the lower side in the drawing) in the direction perpendicular to the optical axis direction of the condenser lens 59 with respect to the combining member 57. The detection unit 63 is provided on the other side (the lower side in the drawing) in the direction perpendicular to the optical axis direction of the condenser lens 59 with respect to the combining member 56.

In the light source device 5 shown in FIG. 25, the combining member 56 has a function of transmitting a part of the blue light BL incident on the one surface 56a in the direction of the detection unit 63 and of reflecting a part of the red light RL incident on the other surface 56b in the direction of the detection unit 63. The combining member 57 has a function of transmitting a part of the violet light VL incident on the one surface 57a in the direction of the detection unit 62 and of reflecting a part of the blue light BL incident on the other surface 57b in the direction of the detection unit 62. The combining member 58 has a function of transmitting the red light WLr and the blue light WLb of the white light WL incident on the one surface 58a in the direction of the detection unit 61 and of reflecting a part of the violet light VL incident on the other surface 58b in the direction of the detection unit 61.

In the light source device 5 having the configuration shown in FIG. 25, in the fifth mode, the processor 5P controls the amount of the red light RL generated by the first light source 51, the amount of the blue light BL generated by the second light source 52, the amount of the violet light VL generated by the third light source 53, and the amount of the white light WL generated by the fourth light source 54, based on the information on the violet light VL, the blue light WLb, and the red light WLr detected by the detection unit 61, the information on the violet light VL and the blue light BL detected by the detection unit 62, and the information on the red light RL and the blue light BL detected by the detection unit 63.

In the above description, the detection unit 60 (the detection unit 61, the detection unit 62, and the detection unit 63) is provided at a position where light reflected by the one surface of the combining member or light transmitted through the other surface can be detected, but the present disclosure is not limited thereto. For example, the detection unit 60 may be provided at a position where light incident on the other surface of the combining member and reflected by the other surface can be detected. In addition, the detection unit 60 may be provided at a position where light before being emitted from the light source and incident on the other surface of the combining member can be detected.

In a case of the example of FIG. 11, a configuration may be employed in which the detection unit 60 that detects leaked light of the violet light VL reflected by the one surface 57a of the combining member 57 or of the violet light VL emitted from the third light source 53 is provided at a position between the third light source 53 and the combining member 57, and the detection unit 60 that detects leaked light of the red light WLr and blue light WLb reflected by the one surface 58a of the combining member 58 or of the red light WLr and blue light WLb emitted from the fourth light source 54 is provided at a position between the fourth light source 54 and the combining member 58.

According to the configurations shown in FIGS. 11, 15 to 19, and 22 to 25, it is possible to inexpensively generate broadband green light GL by using the fourth light source 54 that generates the white light WL, and it is possible to accurately control the amount of light of the light source by using the red light WLr and the blue light WLb included in the white light WL. Since all the green light GL can be introduced into the light guide 14, the quality of the captured image captured by the endoscope 1 can be enhanced.

Next, white balance processing performed by the processor 4P of the processor device 4 will be described.

The processor 4P acquires a captured image signal (a set of pixel signals output from each pixel of the imaging element) output from the imaging element of the endoscope 1 and performs white balance processing on the captured image signal obtained after a demosaicing process of the captured image signal. The white balance processing includes processing of multiplying each pixel signal of each color component included in the captured image signal by an adjustment gain.

For example, in a case where the captured image signal obtained by imaging with the endoscope 1 in a state in which the light source device 5 operates in the first mode is acquired, the processor 4P performs white balance processing by multiplying a pixel signal of a red component included in the captured image signal after the demosaicing process by an adjustment gain Gr1, by multiplying a pixel signal of a green component included in the captured image signal by an adjustment gain Gg1, and by multiplying a pixel signal of a blue component included in the captured image signal by an adjustment gain Gb1.

In addition, in a case where the captured image signal obtained by imaging with the endoscope 1 in a state in which the light source device 5 operates in the second mode is acquired, the processor 4P performs white balance processing by multiplying the pixel signal of the green component included in the captured image signal after the demosaicing process by an adjustment gain Gg2 and by multiplying a pixel signal of a violet component included in the captured image signal by an adjustment gain Gv2.

The processor 4P performs white balance processing by using adjustment gains corresponding to the mode of the light source device 5, for each mode.

In the endoscope apparatus 100, a plurality of types of endoscopes 1 including the light guides 14 with different outer diameters can be connected to the light source device 5 and to the processor device 4. For example, in the endoscope apparatus 100, as the endoscope 1, at least two of a side-viewing endoscope, a duodenoscope, a transnasal endoscope, an oral endoscope, and a bronchoscope can be connected. The outer diameters of the light guides 14 may be different in these endoscopes.

Proper values of the adjustment gains vary depending on the type of the endoscope 1. In a case where the outer diameters of the light guides 14 are different from each other, a positional relationship between the proximal end surface of the light guide 14 and the condenser lens 59 can also be changed. Therefore, the adjustment gain for obtaining appropriate white balance can also be changed. Accordingly, it is preferable that the memory of the processor device 4 stores the adjustment gain for each mode of the light source device 5 in association with the type of the endoscope 1 that can be connected to the light source device 5 and to the processor device 4.

In a case of performing the white balance processing, it is preferable that the processor 4P performs the white balance processing on the captured image signal output from the imaging element of the endoscope 1 connected to the light source device 5 and to the processor device 4 based on the type of the endoscope 1.

Specifically, in a case of performing the white balance processing, the processor 4P recognizes the type of the endoscope 1 connected to the light source device 5 and to the processor device 4, reads out the adjustment gain corresponding to the recognized type from the memory, and performs the white balance processing by using the read-out adjustment gain. By doing so, it is possible to perform the white balance processing suitable for the endoscope 1 in use even in a case where various types of endoscopes 1 are used.

The memory of the processor 4P need not store the adjustment gains at the time of shipment. In this case, the processor 4P can perform appropriate white balance processing by performing data processing of generating the adjustment gain described above.

This data processing is performed, for example, in a state in which a cap including a white image serving as a white reference is attached to the distal end portion 10C of the endoscope 1. In a state in which the cap is attached to the distal end portion 10C, the above-described white image can be captured by the endoscope 1.

In a case where the endoscope 1 is connected, the processor 4P acquires identification information (for example, an individual identification number and a type) for identifying the endoscope 1. In addition, the processor 4P acquires the captured image signal obtained by imaging the white image with the endoscope 1, generates the adjustment gain based on the acquired captured image signal, and stores the adjustment gain in the memory in association with the above-described identification information. The processor 4P performs the data generation processing in a state in which the light source device 5 is operated in each mode, and generates the adjustment gain for each mode of the light source device 5 and stores the adjustment gain in the memory.

As a result, an adjustment gain group corresponding to the first mode, an adjustment gain group corresponding to the second mode, an adjustment gain group corresponding to the third mode, an adjustment gain group corresponding to the fourth mode, and an adjustment gain group corresponding to the fifth mode are stored in the memory of the processor device 4 in association with the identification information of the connected endoscope 1.

In a case where the endoscope 1 whose identification information is stored in the memory is connected, the processor 4P performs the white balance processing by using the adjustment gain corresponding to the identification information that has been stored in the memory. On the other hand, in a case where the endoscope 1 whose identification information is not stored in the memory is connected, the processor 4P performs the data generation processing and performs the white balance processing by using the generated adjustment gain.

In this way, by performing the data generation processing, the processor 4P can generate the adjustment gains suitable for the type of the endoscope 1 to be used even in a case where the types thereof vary, and the quality of the captured image can be enhanced. In addition, even in a case where the same type of endoscope 1 is used, the adjustment gains suitable for each individual endoscope 1 can be generated, and the quality of the captured image can be enhanced. Further, it is not necessary to generate the adjustment gains at the time of manufacturing the processor 4P, and the manufacturing cost can be reduced.

Since the detection unit 60 is provided in the light source device 5 and the light source devices 5B, 5C, 5D, 5F, and 5H, it is possible to suppress changes in the amount of light of each color to be introduced into the light guide 14 due to aging of each light source over time. Therefore, by performing the data generation processing, it is possible to continue using the adjustment gain generated in the data generation processing to enhance the quality of the captured image. On the other hand, a case is also assumed in which the detection unit 60 is not provided in the light source device 5 and the light source devices 5B, 5C, 5D, 5F, and 5H. In this case, in a case where the changes in the amount of light due to aging of each light source over time occur, there is a possibility that the adjustment gain generated once will deviate from the proper value.

In that respect, it is preferable that the processor 4P executes the data generation processing in a case where a predetermined condition related to the operation of the light source device 5 is satisfied, even in a case where the endoscope 1 whose identification information and adjustment gain have been stored is connected. This condition is, for example, that a cumulative value of an operation time of the light source device 5 reaches a predetermined value. The predetermined value is, for example, a value α times (α is a natural number of 1 or more) a default time.

By doing so, it is possible to maintain the white balance properly even in a case where aging occurs over time in the light source device 5. In the light source device 5, in a case where the detection unit 60 is not provided, the manufacturing cost of the light source device 5 can be reduced.

Even in a case where the same outer diameter of the light guide 14 of the endoscope 1 is used, there is a possibility that changes in a color tone of the captured image will occur due to factors such as an arrangement form of the imaging element provided in the distal end portion 10C (for example, whether the light-receiving surface of the imaging element is perpendicular or horizontal to a longitudinal direction of the insertion part 10) and optical characteristics of the imaging optical system. Therefore, even in a configuration in which only the endoscope 1 including the light guide 14 with the same outer diameter can be connected to the endoscope apparatus 100, the processor 4P can generate and store the adjustment gain for each endoscope 1 by performing the data generation processing described above, so that the quality of the image captured by the endoscope 1 can be improved.

In the above description, various types of processing performed by the processor 4P may be performed solely by the processor 4P or may be performed by being shared between the processor 4P and another processor. In addition, various types of processing performed by the processor 5P may be performed solely by the processor 5P or may be performed by being shared between the processor 5P and another processor. The other processor is, for example, a processor of a server in an examination system in which examination data generated by the endoscope apparatus 100 is stored, the processor 4P, the processor 5P, or the like. The various types of processing performed by the processor 5P can also be performed by the processor 4P.

The configurations of the optical member 55 in the light source device 5 and the light source devices 5B, 5C, 5D, 5F, and 5H are examples and are not limited thereto. The optical member 55 can also employ other configurations as long as the optical member 55 is configured to introduce the red light RL into the light guide 14, introduce the blue light BL into the light guide 14, introduce the violet light VL into the light guide 14, and introduce the green light GL into the light guide 14.

As described so far, at least the following matters are described in the present specification. Hereinafter, constituent elements corresponding to the above-described embodiments are shown in parentheses, but the present invention is not limited thereto.

(1)

A system (endoscope apparatus 100) comprising:
a light source device (light source devices 5 and 5D) to which a plurality of types of endoscopes (endoscopes 1) including light guides with different outer diameters are connectable; and
a processor (processor 4P),
in which the light source device includes:
   a first light source (first light source 51) that includes a light emitting diode (light emitting diode 51A) and that generates red light (red light RL);
   a second light source (second light source 52) that includes a light emitting diode (light emitting diode 52A) and that generates blue light (blue light BL);
   a third light source (third light source 53) that includes a light emitting diode (light emitting diode 53A) and that generates violet light (violet light VL);
   a fourth light source (fourth light source 54) that includes a light emitting diode (light emitting diode 54A) and that generates white light (white light WL); and
   an optical member (optical member 55) that is capable of introducing at least two of the red light, the blue light, the violet light, and green light (green light GL) included in the white light into the light guide (light guide 14) of the endoscope by combining the at least two,
the fourth light source generates the white light by using a blue light emitting diode (light emitting diode 54A) that generates blue light and a phosphor (first phosphor 54R and second phosphor 54G, or third phosphor 54Y) that generates light by receiving the blue light,
an optical path of light emitted from the fourth light source to the light guide is shorter than an optical path of light emitted from the first light source, the second light source, and the third light source to the light guide, and
the processor performs white balance processing on a captured image signal output from an imaging element of an endoscope connected to the light source device based on a type of the endoscope.

(2)

The system according to (1),
in which the processor performs data generation processing of generating data to be used for the white balance processing.

(3)

The system according to (1),
in which the plurality of types of endoscopes include at least one of a side-viewing endoscope, a duodenoscope, a transnasal endoscope, or a bronchoscope.

(4)

The system according to (1),
in which the fourth light source generates the white light by using the blue light emitting diode (light emitting diode 54A) that generates blue light, and two types of phosphors (first phosphor 54R and second phosphor 54G) that generate light by receiving the blue light.

(5)

The system according to (4),
in which the two types of phosphors are a first phosphor (first phosphor 54R) that generates red light by receiving the blue light and a second phosphor (second phosphor 54G) that generates green light by receiving the blue light.

(6)

The system according to (2),
in which the processor stores, in a case where the data generation processing is performed, the data generated by the data generation processing in correspondence with the type of the endoscope connected to the light source device.

(7)

The system according to (6),
in which the processor performs the data generation processing in a case where an endoscope of a type whose data is not stored is connected.

(8)

The system according to (6),
in which the processor performs the data generation processing in a case where a predetermined condition related to an operation of the light source device is satisfied.

(9)

The system according to (2),
in which the light source device is operable in a plurality of modes in which the number of rays of light to be introduced into the light guide of the endoscope varies, and
the processor performs the data generation processing for each of the plurality of modes.

(10)

The system according to (9),
in which the plurality of modes include a first mode in which the red light, the blue light, and the green light are combined and introduced into the light guide.

(11)

The system according to (10),
in which the plurality of modes include a second mode in which the violet light and the green light are combined and introduced into the light guide.

(12)

The system according to (1),
in which the light source device includes a detection unit (detection unit 60) that detects a part of light generated by the first light source, the second light source, the third light source, and the fourth light source, and the light source device controls amounts of light generated by at least two of the first light source, the second light source, the third light source, and the fourth light source based on the light detected by the detection unit.

(13)

A light source device (light source devices 5, 5B, 5D, 5F, and 5H) comprising:
a first light source (first light source 51) that includes a light emitting diode (light emitting diode 51A) and that generates red light (red light RL);
a second light source (second light source 52) that includes a light emitting diode (light emitting diode 52A) and that generates blue light (blue light BL);
a third light source (third light source 53) that includes a light emitting diode (light emitting diode 53A) and that generates violet light (violet light VL);
a fourth light source (fourth light source 54) that includes a light emitting diode (light emitting diode 54A) and that generates white light (white light WL);
an optical member (optical member 55) that is configured to be capable of introducing the red light into a light guide (light guide 14) of an endoscope (endoscope 1), introducing the blue light into the light guide, introducing the violet light into the light guide, and introducing green light (green light GL) included in the white light into the light guide;
a detection unit (detection unit 60) that detects a part of light generated by at least two of the first light source, the second light source, the third light source, and the fourth light source; and
a processor (processor 5P) that controls amounts of light generated by at least two of the first light source, the second light source, the third light source, and the fourth light source based on the light detected by the detection unit.

(14)

The light source device according to (13),
in which a light source whose amount of light is controlled by the processor based on the light detected by the detection unit includes the third light source.

(15)

The light source device according to (14),
in which a light source whose amount of light is controlled by the processor based on the light detected by the detection unit includes the fourth light source.

(16)

The light source device according to (15),
in which the detection unit detects the violet light and blue light (blue light WLb) included in the white light, and
the processor controls amounts of light of the third light source and the fourth light source based on the detected violet light and blue light.

(17)

The light source device according to (14),
in which a light source whose amount of light is controlled by the processor based on the light detected by the detection unit includes the second light source.

(18)

The light source device according to (14),
in which the optical member includes a first combining member (combining member 58 in FIGS. 11 to 17 and 22 to 25 and combining member 57 in FIGS. 18 and 19) that is capable of combining the violet light and the green light.

(19)

The light source device according to (18),
in which the detection unit includes a first detection unit (detection unit 61 in FIGS. 11 to 17 and 22 to 25 and detection unit 62 in FIGS. 18 and 19) that detects a part of light incident on the first combining member.

(20)

The light source device according to (19),
in which the optical member includes a second combining member (combining member 56 in FIGS. 11 to 17 and 22 to 25) that is capable of combining a plurality of rays of light and a third combining member (combining member 57 in FIGS. 11 to 17 and 22 to 25) that is capable of combining a plurality of rays of light, and
the first combining member is disposed closer to the light guide than the second combining member and the third combining member.

(21)

The light source device according to any one of (18) to (20),
in which the detection unit detects a part of the violet light incident on the first combining member and blue light (blue light WLb) included in the white light incident on the first combining member, and
the processor controls an amount of light of the third light source and an amount of light of the fourth light source based on the detected violet light and blue light.

(22)

The light source device according to (14),
in which the optical member includes three combining members (combining member 56, combining member 57, and combining member 58) that are capable of combining a plurality of rays of light, and
the detection unit detects a part of light incident on any of two combining members (combining member 57 and combining member 58) excluding the combining member (combining member 56) located at an end opposite to a side of the light guide among the three combining members.

(23)

The light source device according to (22),
in which the detection unit detects a part of light incident on the combining member (combining member 58) located at an end on the side of the light guide among the three combining members.

(24)

The light source device according to any one of (13) to (17),
in which the number of the detection units is less than a total number of the first light source, the second light source, the third light source, and the fourth light source.

(25)

The light source device according to (24),
in which the number of the detection units is one.

(26)

The light source device according to (25),
in which the optical member includes a first combining member (combining member 58 in FIGS. 11 to 17 and combining member 57 in FIGS. 18 and 19) that is capable of combining the violet light and the green light, and
the detection unit detects a part of light incident on the first combining member.

(27)

The light source device according to (26),
in which the optical member includes a second combining member (combining member 56 in FIGS. 11 to 17) that is capable of combining a plurality of rays of light and a third combining member (combining member 57 in FIGS. 11 to 17) that is capable of combining a plurality of rays of light, and
the first combining member (combining member 58 in FIGS. 11 to 17) is disposed closer to the light guide than the second combining member and the third combining member.

(28)

The light source device according to (27),
in which the processor controls an amount of light generated by the first light source, an amount of light generated by the second light source, an amount of light generated by the third light source, and an amount of light generated by the fourth light source based on the light detected by the detection unit.

(29)

The light source device according to any one of (13) to (20), (22), and (23),
in which the fourth light source includes a blue light emitting diode (light emitting diode 54A) that generates blue light, a first phosphor (first phosphor 54R) that generates red light by receiving the blue light generated by the blue light emitting diode, and a second phosphor (second phosphor 54G) that generates green light by receiving the blue light generated by the blue light emitting diode.

(30)

The light source device according to any one of (13) to (20), (22), and (23),
in which the fourth light source includes a blue light emitting diode (light emitting diode 54A) that generates blue light and a phosphor (third phosphor 54Y) that generates yellow light by receiving the blue light generated by the blue light emitting diode. (31)

The light source device according to any one of (13) to (20), (22), and (23),
in which the processor selectively performs a first control of introducing the red light, the blue light, and the green light (green light GL) included in the white light into the light guide, and a second control of introducing the violet light and the green light (green light GL) included in the white light into the light guide.

(32)

The light source device according to any one of (13) to (20), (22), and (23),
in which the light generated by the third light source has a central wavelength of 410nm or more and 430 nm or less and a half-width of 15 nm or more and 30 nm or less. (33)

The light source device according to any one of (13) to (20), (22), and (23),
in which the blue light is generated by the light emitting diode (light emitting diode 52A) provided in the second light source, and
the light generated by the light emitting diode has a central wavelength of 440 nm or more and 450 nm or less and a half-width of 15 nm or more and 30 nm or less. (34)

The light source device according to any one of (13) to (20), (22), and (23),
in which the processor performs a control of introducing the violet light and the green light (green light GL) included in the white light into the light guide, and
in the control, the green light introduced into the light guide has a central wavelength of 540 nm or more and 560 nm or less and a half-width of 80 nm or more.

(35)

A light source device (light source devices 5 and 5B to 5C) comprising:
a first light source (first light source 51) that includes a light emitting diode (light emitting diode 51A) and that generates first color light (red light RL);
a second light source (second light source 52) that includes a light emitting diode (light emitting diode 52A) and that generates second color light (blue light BL);
a third light source (third light source 53) that includes a light emitting diode (light emitting diode 53A) and that generates violet light (violet light VL);
a fourth light source (fourth light source 54, fourth light source 54X, and fourth light source 54Xa) that includes a light emitting diode (light emitting diode 54A) and that generates light including at least green light;
a first combining member (combining member 56 in FIGS. 11 to 15, 23, and 25) that is capable of combining the first color light and the second color light;
a second combining member (combining member 57 in FIGS. 11 to 15, 23, and 25) that is capable of combining light emitted from the first combining member and one of the violet light and the green light;
a third combining member (combining member 58 in FIGS. 11 to 15, 23, and 25) that is capable of combining light emitted from the second combining member and the other of the violet light and the green light;
a condensing member (condenser lens 59) that condenses light emitted from the third combining member into a light guide (light guide 14) of an endoscope (endoscope 1);
a first detection unit (detection unit 61 in FIGS. 11 to 15, 23, and 25) that detects a part of light emitted from the second combining member and incident on the third combining member and a part of light emitted from the fourth light source and incident on the third combining member; and
a processor (processor 5P) that controls amounts of light generated by at least two of the first light source, the second light source, the third light source, and the fourth light source based on the light detected by the first detection unit.

(36)

The light source device according to (35),
in which the fourth light source (fourth light source 54) generates white light (white light WL) including the green light.

(37)

The light source device according to (36),
in which the fourth light source (fourth light source 54) includes a blue light emitting diode (light emitting diode 54A) that generates blue light, a first phosphor (first phosphor 54R) that generates red light by receiving the blue light generated by the blue light emitting diode, and a second phosphor (second phosphor 54G) that generates the green light by receiving the blue light generated by the blue light emitting diode.

(38)

The light source device according to (36) or (37),
in which the first detection unit detects the violet light and blue light (blue light WLb) included in the white light.

(39)

The light source device according to (38),
in which the processor controls amounts of light of the third light source and the fourth light source based on the violet light and the blue light detected by the first detection unit.

(40)

The light source device according to (35),
in which the fourth light source (fourth light source 54Xa) includes a blue light emitting diode (light emitting diode 54A) that generates blue light and a third phosphor (fourth phosphor 54Gx) that generates the green light by receiving the blue light generated by the blue light emitting diode.

(41)

The light source device according to any one of (35) to (37), further comprising:
a second detection unit (detection unit 63 in FIGS. 23 and 25) that detects a part of light incident on the first combining member.

(42)

The light source device according to (41),
in which the second detection unit detects the first color light and the second color light, and
the processor controls amounts of light of the first light source and the second light source based on the first color light and the second color light detected by the second detection unit.

(43)

The light source device according to (41), further comprising:
a third detection unit (detection unit 62 in FIG. 25) that detects a part of light emitted from the first combining member and incident on the second combining member and a part of light emitted from the third light source and incident on the second combining member.

(44)

The light source device according to (43),
in which the processor controls an amount of light generated by the first light source, an amount of light generated by the second light source, an amount of light generated by the third light source, and an amount of light generated by the fourth light source based on the light detected by the first detection unit, the second detection unit, and the third detection unit.

### EXPLANATION OF REFERENCES

1: endoscope

2: body part

4: processor device

4P: processor

5, 5B, 5C, 5D, 5F, 5H: light source device

5P: processor

6: input unit

7: display device

10: insertion part

10A: soft portion

10B: bending portion

10C: distal end portion

11: operation part

12: angle knob

13: universal cord

13A, 13B: connector portion

14: light guide

51: first light source

51A, 52A, 53A, 54A, 54g: light emitting diode

52: second light source

53: third light source

54, 54X, 54Xa: fourth light source

54F: excitation light cut filter

54R: first phosphor

54G: second phosphor

54Y: third phosphor

54Gx: fourth phosphor

55: optical member

56, 57, 58: combining member

56a, 57a, 58a: one surface

56b, 57b, 58b: other surface

59: condenser lens

60, 61, 62, 63: detection unit

100: endoscope apparatus

RL, WLr: red light

BL, WLb, B1: blue light

GL: green light

VL: violet light

WL: white light

What is claimed is:

1. A system comprising:
a light source device to which a plurality of types of endoscopes, each of which includes a light guide having a different outer diameter from an outer diameter of the light guide included in other of the plurality of types of endoscopes, are connectable; and
a processor,
wherein the light source device includes:
a first light source that includes a light emitting diode and that generates red light;
a second light source that includes a light emitting diode and that generates blue light;
a third light source that includes a light emitting diode and that generates violet light;
a fourth light source that includes a light emitting diode and that generates white light; and
an optical member that is capable of introducing at least two of the red light, the blue light, the violet light, and green light included in the white light into the light guide of the endoscope by combining the at least two,
the fourth light source generates the white light by using a blue light emitting diode that generates blue light and a phosphor that generates light by receiving the blue light,
an optical path of light emitted from the fourth light source to the light guide is shorter than an optical path of light emitted from the first light source to the light guide, is shorter than an optical path of light emitted from the second light source to the light guide, and is shorter than an optical path of light emitted from the third light source to the light guide, and
the processor is configured to perform white balance processing on a captured image signal output from an imaging element of one of the endoscopes connected to the light source device based on a type of the one of the endoscopes.

2. The system according to claim 1,
wherein the processor is configured to perform data generation processing of generating data to be used for the white balance processing.

3. The system according to claim 1,
wherein the plurality of types of endoscopes include at least one of a side-viewing endoscope, a duodenoscope, a transnasal endoscope, or a bronchoscope.

4. The system according to claim 1,
wherein the fourth light source generates the white light by using the blue light emitting diode that generates blue light, and two types of phosphors that generate light by receiving the blue light.

5. The system according to claim 4,
wherein the two types of phosphors are a first phosphor that generates red light by receiving the blue light and a second phosphor that generates green light by receiving the blue light.

6. The system according to claim 2,
wherein the processor is configured to store, in a case where the data generation processing is performed, the data generated by the data generation processing in correspondence with the type of the endoscope connected to the light source device.

7. The system according to claim 6,
wherein the processor is configured to perform the data generation processing in a case where the endoscope of a type, with which the data is not stored in correspondence, is connected to the light source device.

8. The system according to claim 6,
wherein the processor is configured to perform the data generation processing in a case where a predetermined condition related to an operation of the light source device is satisfied.

9. The system according to claim 2,
wherein the light source device is operable in a plurality of modes in which number of rays of light to be introduced into the light guide of the endoscope varies, and
the processor is configured to perform the data generation processing for each of the plurality of modes.

10. The system according to claim 9,
wherein the plurality of modes include a first mode in which the red light, the blue light, and the green light are combined and introduced into the light guide.

11. The system according to claim 10,
wherein the plurality of modes include a second mode in which the violet light and the green light are combined and introduced into the light guide.

12. The system according to claim 1,
wherein the light source device includes a detection unit that detects a part of light generated by the first light source, the second light source, the third light source, and the fourth light source, and the light source device controls amounts of light generated by at least two of the first light source, the second light source, the third light source, and the fourth light source based on the light detected by the detection unit.

13. A light source device comprising:
a first light source that includes a light emitting diode and that generates first color light;
a second light source that includes a light emitting diode and that generates second color light;
a third light source that includes a light emitting diode and that generates violet light;
a fourth light source that includes a light emitting diode and that generates light including at least green light;
a first combining member that is capable of combining the first color light and the second color light;
a second combining member that is capable of combining light emitted from the first combining member and one of the violet light and the green light;
a third combining member that is capable of combining light emitted from the second combining member and other of the violet light and the green light;
a condensing member that condenses light emitted from the third combining member into a light guide of an endoscope;
a first detection unit that detects a part of light emitted from the second combining member and incident on the third combining member and a part of light emitted from the fourth light source and incident on the third combining member; and
a processor that is configured to control amounts of light generated by at least two of the first light source, the second light source, the third light source, and the fourth light source based on the light detected by the first detection unit.

14. The light source device according to claim 13,
wherein the fourth light source generates white light including the green light.

15. The light source device according to claim 14,
wherein the fourth light source includes a blue light emitting diode that generates blue light, a first phosphor that generates red light by receiving the blue light generated by the blue light emitting diode, and a second phosphor that generates the green light by receiving the blue light generated by the blue light emitting diode.

16. The light source device according to claim 14,
wherein the first detection unit detects the violet light and blue light included in the white light.

17. The light source device according to claim 16,
wherein the processor is configured to control amounts of light of the third light source and the fourth light source based on the violet light and the blue light detected by the first detection unit.

18. The light source device according to claim 13,
wherein the fourth light source includes a blue light emitting diode that generates blue light and a third phosphor that generates the green light by receiving the blue light generated by the blue light emitting diode.

19. The light source device according to claim 13, further comprising:
a second detection unit that detects a part of light incident on the first combining member.

20. The light source device according to claim 19,
wherein the second detection unit detects the first color light and the second color light, and
the processor is configured to control amounts of light of the first light source and the second light source based on the first color light and the second color light detected by the second detection unit.

21. The light source device according to claim 19, further comprising:
a third detection unit that detects a part of light emitted from the first combining member and incident on the second combining member and a part of light emitted from the third light source and incident on the second combining member.

22. The light source device according to claim 21,
wherein the processor is configured to control an amount of light generated by the first light source, an amount of light generated by the second light source, an amount of light generated by the third light source, and an amount of light generated by the fourth light source based on the light detected by the first detection unit, the second detection unit, and the third detection unit.
